(19) United States
(12) Patent Application Publication
(10) Pub. No.: US 2022/0347320 A1
(43) Pub. Date: Nov. 3, 2022

(54) REGENERATION OF RETINAL GANGLION CELLS

(71) Applicant: SHANGHAITECH UNIVERSITY, Shanghai (CN)

(72) Inventors: Hongjun Liu, Shanghai (CN); Xiaohu Wei, Shanghai (CN); Na Qiao, Shanghai (CN)

(21) Appl. No.: 17/812,381

(22) Filed: Jul. 13, 2022

Related U.S. Application Data
(63) Continuation of application No. PCT/CN2021/072108, filed on Jan. 15, 2021.

(30) Foreign Application Priority Data
Jan. 16, 2020 (CN) 202010047628.2

(51) Int. Cl. A61K 48/00; C12N 15/86; A61K 38/17; A61P 27/02

(52) U.S. Cl. CPC A61K 48/0075; C12N 15/86; A61K 38/1709; A61P 27/02; C12N 2750/14023; C12N 2750/14142; C12N 2750/14133

(57) ABSTRACT

Provided herein are compositions and methods for regenerating retinal ganglion cells (RGCs) from retinal neuron cells by activating transcription factors such as one or more of Atoh7, Brn3B, Sox4, Sox11, or Ils1. The retinal neuron cells may be interneuron cells such as amacrine cells, horizontal cells, and bipolar cell. The regenerated RGCs can project axons into discrete subcortical brain regions and establish retina-brain connections. They can respond to visual stimulation and transmit electrical signals into the brain. Therefore, the regenerated RGCs can replace damaged or degenerated RGCs, thereby treating vision impairment or blindness. The methods are likewise applicable to degenerated, damaged, or aged RGCs to stimulate them to regrow functional axons, thereby rejuvenating these RGCs.

### CROSS-REFERENCE TO RELATED PATENT APPLICATIONS

This application is a continuation application of International Application No. PCT/CN2021/072108, filed Jan. 15, 2021, which claims the priority to Chinese Patent Application No. 202010047628.2, filed Jan. 16, 2020, the contents of each of which are hereby incorporated by reference in their entirety into the present disclosure.

### REFERENCE TO AN ELECTRONIC SEQUENCE LISTING

The contents of the electronic sequence listing (326023.xml; Size: 41,368 bytes; and Date of Creation: Jul. 12, 2022) is herein incorporated by reference in its entirety.

### BACKGROUND

Retinal ganglion cells (RGCs) are the final output neurons of the retina that process visual information and transmit it to discrete brain visual areas to form vision. Loss of RGCs is a leading cause of blindness in a group of diseases broadly categorized as optic neuropathies, including glaucoma, hereditary optic neuropathies, and disorders caused by toxins, nutritional defects and trauma. Vision loss in these patients is irreversible since humans and all mammals lack the ability to generate RGCs in adulthood. There is great interest in developing regenerative therapies to restore lost vision in such patients.

One attractive approach of developing regenerative therapies for optic neuropathies is to replace lost ganglion cells and reconnect the retina to the brain using endogenous cells. Tremendous efforts have been made to identify retinal stem/progenitor cells and to understand how retinal neurons are generated in a variety of model organisms. Previous studies demonstrated that lower vertebrates, like fish and amphibians, functionally regenerate their retinas following injury, and Müller glia are the cellular source of regenerated retinal neurons. By contrast, Müller glia in mammals do not have this capacity and mammals, including humans, also do not have other reservoirs of retinal stem/progenitor cells poised to regenerate retinal neurons in the adult stage. The current consensus is that there is normally little to no ongoing addition of neurons in the mature mammalian retina.

There is a strong need to treat these diseases and conditions and restore the vision of the patients.

### SUMMARY

The present disclosure reports the discovery that retinal ganglion cells (RGCs) can be regenerated from retinal neurons by activating transcription factors such as one or more of Atoh7, Brn3B, Sox4, Sox11, or Ils1. The regenerated RGCs can project axons into discrete subcortical brain regions and establish retina-brain connections. They can respond to visual stimulation and transmit electrical signals into the brain. Therefore, the regenerated RGCs can replace damaged or degenerated RGCs, thereby treating vision impairment or blindness.

In another unexpected discovery, activation of these transcription factors can also reactivate degenerated, damaged, or aged RGCs so that they can regrow functional axons. Accordingly, when therapeutic agents that can activate these transcription factors are administered to a subject, they can rejuvenate degenerated, damaged, or aged RGCs, and the same time reprogram the nearby interneuron cells into regenerated RGCs. Such dual effects of these agents can be more effective in achieving the desired therapeutic effect.

In accordance with one embodiment of the present disclosure, provided is a method for preparing a mammalian cell responsive to visual signals, comprising increasing the biological activity, a retinal neuron cell, of one or more genes selected from the group consisting of: POU class 4 homeobox 2 (Brn3B), SRY-box transcription factor 4 (Sox4), Atonal BHLH Transcription Factor 7 (Atoh7), SRY-Box Transcription Factor 11 (Sox11), and ISL LIM homeobox 1 (Ils1).

In some embodiments, the one or more genes comprise Brn3B and Sox4. In some embodiments, the one or more genes further comprise Atoh7.

In some embodiments, the retinal neuron cell is an interneuron cell, such as an amacrine cell, a horizontal cell, or a bipolar cell. In some embodiments, the retinal neuron cell is a degenerated, damaged, or aged retinal ganglion cell (RGC). In some embodiments, the retinal neuron cell is a Lgr5⁺ amacrine cell. In some embodiments, the retinal neuron cell is a Prokr2⁺ displaced amacrine cell.

In another embodiment, the present disclosure provides a method for improving the function of a retinal ganglion cell (RGC), which may be a degenerated, damaged, aged, or a normal/healthy, for which improved function is desired. In some embodiments, the method entails increasing the biological activity, in the RGC, of one or more genes selected from the group consisting of Atoh7, Brn3B, Sox4, Sox11, and Ils1.

In some embodiments, increasing the biological activity of the one or more genes comprises introducing to the retinal neuron cell one or more polynucleotide encoding the genes, such as cDNA, which can be provided in a plasmid or viral vector, such as an adeno-associated viral (AAV) vector.

Also provided is a method for treating visual impairment or blindness in a subject in need thereof, comprising administering to the retina of the subject an agent capable of increasing the biological activity of one or more genes selected from the group consisting of Brn3B, Sox4, Atoh7, Sox11, and Ils1.

In some embodiments, the visual impairment or blindness is caused by degenerated retinal ganglion cells (RGCs). In some embodiments, the visual impairment or blindness is associated with a condition selected from the group consisting of optic neuropathy, including glaucoma, hereditary optic neuropathy, and disorders caused by toxins, nutritional defects and trauma.

Also provided, in one embodiment, is a nucleic acid construct comprising coding sequences encoding the Brn3B and Sox4 proteins, and a promoter associated with each coding sequence, wherein each promoter is active in retinal neuron cells.

Another embodiment provides a cell transfected by the nucleic acid construct. Yet another embodiment provides a cell responsive to visual signals, prepared by the instantly disclosed methods.

These and other embodiments are further described in the text that follows.

### BRIEF DESCRIPTION OF THE DRAWINGS

FIG. 1. Lgr5⁺ amacrine interneurons transdifferentiate into other neuronal subtypes in adult mice. a, Image of retina cross section showing Lgr5⁺ amacrine interneurons in the inner nuclear layer. b, c, Images of retina cross sections from Lgr5^{EGFP-IRES-CreERT2} Rosa26-tdTomato mice. Arrows highlight generation of bipolar (b) and horizontal (c) cells from Lgr5⁺ amacrine interneurons. d, Image of flat-mount retina sample, focusing on the retinal ganglion cell layer. Lgr5⁺ amacrine interneurons that have migrated from the inner nuclear layer to the ganglion cell layer are labeled in green. e, Representative membrane potential of Lgr5⁺ amacrine cells in response to full field light flash. Inset: fluorescent image of the recorded cell after dye filling. f, Representative excitatory postsynaptic current (EPSC, blue) and inhibitory postsynaptic current (IPSC, red) of Lgr5⁺ amacrine cells in response to full field light flash. In total, 5 out of 6 recorded cells showed responses to full field LED light stimulation. Arrows in panels e and f: stimulus artifact. ONL: outer nuclear layer; OPL: outer plexiform layer; INL: inner nuclear layer; IPL: inner plexiform layer; GCL: ganglion cell layer. Scale bars=30 μm in panels a, b, and c, =200 μm in panel d, and =10 μm in panel e.

FIG. 2. Reprogram Lgr5⁺ amacrine interneurons into RGCs in vivo. a, Strategy of in vivo neuronal reprogramming. Lgr5^{EGFP-IRES-CreERT2} Rosa26-tdTomato mice were first fed with tamoxifen (TM) five times (from day −11 (D-11) to day −7 (D-7)) to label Lgr5⁺ amacrine interneurons with the Rosa26-tdTomato reporter to assist identity tracing. One week later, mice were intravitreally injected with AAVs expressing Cre-dependent transcription factors on D1, and followed with TM feeding from D3 to D7 to activate AAV-delivered genes specifically in Lgr5⁺ amacrine interneurons. Mice were sacrificed for analysis 6 weeks after viral injection. b, Diagrams of AAV expression vectors using the Cre-dependent direction-inverted open reading frame (DIO) system. c, Reprogramming efficiencies of single and various combination of transcription factors. Data presented are numbers of tdTomato⁺ axons in optic nerves (n=8 from 4 mice in each group). No tdTomato⁺ axons could be detected in optic nerves of mice injected with AAV-DIO-EGFP. (D-F) Images of flat-mount retina samples from experimental mice, focusing on the retinal ganglion cell layer. e, High-magnification view of an area in panel D, with arrows pointing to tdTomato⁺ axons of regenerated RGCs. f, Highlight of a single regenerated RGC. g-i, Immunohistological stainings of regenerated RGCs with antibodies specific for RPBMS (g), Brn3A (h) and CART (i). **P<0.001; NS, not significant. Abbreviations in panel c: B=Brn3B, S4=Sox4, BS4=Brn3B+Sox4, ABS4=Atoh7+Bm3B+Sox4, ABS4S11Is=Atoh7+Brn3B+Sox4+Sox11+Isl1. Scale bars=200 μm in panel d, =150 μm in panel e, =50 μm in panel f, and =40 μm in panels g, h and i.

FIG. 3. Regenerated RGCs project axons into the brain. a, Confocal image of axons of regenerated RGCs within the optic nerve. b-f, Projections of regenerated RGC axons in brain visual areas, showing tdTomato⁺ axon terminals in dorsal and ventral lateral geniculate nucleus (dLGN and vLGN, panels b and c respectively), olivary pretectal nucleus (OPN, panel d), and the superior colliculus (SC, panels e and f). Arrows in panel f highlight bouton-like structures on regenerated RGC axon terminals in the SC region. g-i, PSD-95 staining of SC brain sections. tdTomato⁺ varicosities are in close apposition to PSD-95 staining but do not overlap. Brain samples from 3 different animals have the same staining pattern. Scale bars=200 μm in panels a to e, =40 μm in panel f, and =10 μm in panels g and h.

FIG. 4. Reprogram Prokr2⁺ displaced amacrine interneurons into RGCs. a, Confocal image of flat-mount retina sample from Prokr2^{CreERT2}; Rosa26-tdTomato mice. tdTomato⁺ displaced amacrine cells are shown in red. Prokr2-tdTomato⁺ displaced amacrine cells do not have any axons on flat-mount retina sample. b, Highlight of an area in panel a. c, Confocal image of flat-mount retina sample from Prokr2^{CreERT2} mice injected with AAVs co-expressing transcription factors and EGFP. Regenerated RGCs extend axons to the optic disc. d, Highlight of an area in panel c. e, Axons of regenerated RGCs within optic nerve. f-k, Projections of regenerated RGC axons to various brain retinorecipient areas, including dLGN and vLGN (f), OPN (g), and upper (panels h and i) and lower layer (panels j and k) of SC regions Images in panels i and k are high-magnification views. Scale bars=1000 μm in panels a and c, =200 μm in panels e to g, and panel j, =100 μm in panel h, and =40 μm in panels i and k.

FIG. 5. Regenerated RGCs transmit visual information to the brain and establish functional connections with postsynaptic neurons. a, Traces of calcium signals of three example axonal terminals in response to drifting gratings of different directions. Cyan patches mark periods of stimulus presentation, and the values on bottom indicate stimulus direction. Note that the three terminals shown in this panel have robust “on” responses. b, Same plots as in panel A except that terminals shown here have robust “off” responses. c, d, Traces of calcium signals of three axon terminals that have orientation selectivity (c) and direction selectivity (d). e, An representative EPSC of light-evoked postsynaptic AMPA receptor responses in a SC neuron. Arrows indicate the postsynaptic response of multi-presynaptic inputs. f, An representative EPSC of light-evoked postsynaptic NMDA receptor response in a SC neuron. g, An example of light-evoked postsynaptic action potential in a SC neuron. h, i, Summary of EPSC amplitudes (h) and peak numbers (i) of light-evoked AMPA receptor responses (n=11 from 7 animals) j, Summary of EPSC amplitudes of light-evoked NMDA receptor responses (n=3 from 3 animals) Data are mean±sem.

FIG. 6. Regenerate functional RGCs in a mouse model of glaucoma. a-d. Confocal images of retina samples. a, Retina of normal Lgr5^{EGFP-IRES-CreERT2}; Rosa26-tdTomato mice. b, Retina of mice damaged by intraocular pressure increase (IPI) seven days ago. c, Retina of mice damaged by IPI seven days ago but received daily Ripasudil treatment. d, Retina of mice damaged by IPI but received both Ripasudil treatment and injection of AAVs expressing RGC fate-specification transcription factors (AAV-DIO-TFs). Mice were sacrificed 6 weeks after AAV injection. e, f, Confocal images of optic nerves from eyes receiving AAV-DIO-EGFP (e) and AAV-DIO-TFs (f). g-k, Confocal images of brain sections from Lgr5^{EGFP-IRES-CreERT2}; Rosa26-tdTomato mice, which had received injections of AAV-DIO-EGFP in the left eye and AAV-DIO-TFs in the right eye. The majority of regenerated RGC axons were projected to the contralateral (left) side of the brain, with a small portion to the ipsilateral (right) side. Brain visual areas presented are optic track immediately after the optic chiasma (g), optic track (h), dLGN, vLGN and projection to the pretectal areas (i), dLGN (j), and SC (k). l-n, Light-evoked postsynaptic responses of SC neurons, including AMPA receptor-mediated EPSC (1), NMDA receptor-mediated EPSC (n), and action potential (n). Scale bars=100 μm in panels d, j and k, =200 μm in panels e and f, =400 μm in panel g, and =600 μm in panels h and i.

FIG. 7. Morphology of Lgr5⁺ amacrine interneurons and their migration to the ganglion cell layer. a-c, Confocal images of Lgr5⁺ amacrine interneurons sparsely labeled with the tdTomato reporter in Lgr5^{EGFP-IRES-CreERT2}; Rosa26-tdTomato mice. Sparse labelling of Lgr5⁺ amacrine cells with the tdTomato reporter was achieved by feeding mice with Tamoxifen only once. Images were taken from flat-mounted retina samples, focusing on the inner nuclear layer where Lgr5⁺ amacrine cells are localized. d, Confocal images of a retinal cross section from Lgr5^{EGFP-IRES-CreERT2}; Rosa26-tdTomato mice. Arrows highlights a Lgr5⁺ amacrine cell labeled with the tdTomato reporter. The dendritic processes of this cell reach the ganglion cell layer. e-g, Confocal images of flat-mounted retina samples from Lgr5^{EGFP-IRES-CreERT2} mice, focusing on the ganglion cells layer. Arrows highlight the presence of Lgr5⁺ amacrine cells in the ganglion cells layer. g, A group of Lgr5⁺ amacrine cells in the ganglion cell layer of a 20 month-old mouse. h, Number of Lgr5⁺ amacrine cells in the ganglion cell layer per retina. There is an age-dependent increase of Lgr5⁺ amacrine cells in the ganglion cell layer, suggesting that these cells might migrate from the inner nuclear layer to the ganglion cell layer. Scale bars=20 μm in panels a to c, =30 μm in panel d, and =50 μm in panels e to f.

FIG. 8. Neuronal identity reprogramming in Lgr5^{EGFP-IRES-CreERT2}; Rosa26-tdTomato mice. a, b, Representative images of flat-mount retina sample (a) and optic nerve (b) from mice injected with AAV-DIO-EGFP. No tdTomato⁺ axons could be detected in these mice. c-e, Representative images of optic nerve from mice injected with high dose of AAV-DIO-EGFP (7×10¹² pfu, 2 μl). Due to AAV-DIO plasmid self-recombination during DNA amplification and viral vector production (flipped vectors), a small number (within single digit) of original RGCs and their axons could be labeled by injected AAV-DIO-EGFP through Cre-independent transgene expression, when large amount of AAV particles are injected. However, these EGFP⁺ axons do not express tdTomato, suggesting that they are not from regenerated RGCs. f-h, Highlights of a Lgr5-EGFP and tdTomato double positive cells present in the inner plexiform layer (IPL), suggesting that programming triggers migration of Lgr5⁺ amacrine cells from the inner nuclear layer (INL) to the retinal ganglion layer (RGL). i-l, Confocal images of a regenerated RGC expression the alpha-RGC marker SMI-32. m-r, Confocal images of retina cross sections from Lgr5^{EGFP-IRES-CreERT2} mice intravitreally injected with AAV-DIO-tdTomato to examine the specificity and efficiency of AAV delivered gene expression. After 5 tamoxifen feedings, AAV-delivered tdTomato gene specifically label Lgr5⁺ amacrine cells. p-r, Higher magnification images taken from the same eye of panel m to o. s, Statistics of EGFP⁺/tdTomato⁺ cells in panels m to r. With this expression system, about 21.3% Lgr5⁺ amacrine cells could be labeled by AAV delivered tdTomato. t, Statistics of Brn3B and Sox4 expression levels in Lgr5^{EGFP-IRES-CreERT2}; Rosa26-tdTomato mice intravitreally injected with AAV-DIO-Brn3B and AAV-DIO-Sox4. Brn3B and Sox4 expression was measured by quantitative PCR and were normalized to 1 in control mice intravitreaaly injected with AAV-DIO-EGFP. Scale bars=150 μm in panel a, =200 μm in panel b, =300 μm in panels c to e, =40 μm in panels f to l, =400 μm in panel o, and =50 μm in panel r.

FIG. 9. Time taken by regenerated RGCs to grow axons into discrete brain visual areas. Lgr5^{EGFP-IRES-CreERT2}; Rosa26-tdTomato mice were intravitreally injected with AAV-DIO-Brn3B and AAV-DIO-Sox4 in one eye, and were subsequently fed with tamoxifen 5 times to activate gene expression. Mice were sacrificed at different time points for brain slice preparation, and the presence of tdTomato⁺ axons were examined by confocal microscope. a, Confocal image of brain slice from a mouse sacrificed 30 days after viral injection. tdTomato⁺ axons in the contralateral brain side (left side in the picture) has passed the lateral geniculate nucleus. No tdTomato⁺ axons were detected on the ipsilateral side of the same brain slide. b, Confocal image of the superior colliculus (SC) area from the same mouse as in panel a. No tdTomato⁺ axons had reached SC at this time. c, Confocal image of brain slide from a mouse sacrificed 35 days after viral injection. Highlighted are areas in the back of LGN (lateral geniculate nucleus) and the front of SC in the contralateral brain side (left side in the picture). tdTomato⁺ axons could also be detected in the ipsilateral brain side, but the number was dramatically lower. d, Time course of axonal projection of regenerated RGCs. Time taken by regenerated RGC axons to reach OC (optic chiasma), LGN and SC was determined by counting the time when regenerated RGC axons were first observed in these locations after viral injection. Regenerated RGC axons reach OC approximately at day 18, LGN at day 28 and SC at day 35 (n=8 in each group).

FIG. 10. Construction of the Prokr2 knock-in mouse strain and reprogramming Prokr2⁺ displaced amacrine interneurons into RGCs in vivo. a, Diagram of targeting strategy for making the Prokr2^{CreERT2} mouse strain. CreERT2 coding region is knocked into the start codon of the Prokr2 locus. Locations of Southern Blotting probes targeting the 5′ arm and the CreERT2 region were marked. b, Images of Southern Blotting membranes with 5′ arm probe (upper) and CreERT2 probe (lower). Founder 1, 2, 3 and 4 have the correct genome targeting, and were used for further breeding. c-e, Immuno-histological staining of retina cross section from Prokr2^{CreERT2}; Rosa26-tdTomato mice with anti-RBPMS antibody. Prokr2-tdTomato⁺ cells do not express the RGC marker RBPMS. f-h, Confocal images of brain coronal section (f), superior colliculus (g) and optic nerve (h) of Prokr2^{CreERT2}; Rosa26-tdTomato mice. Prokr2-tdTomato⁺ cells are present in the brain and the optic nerve. i, j, Images of Prokr2^{CreERT2} mice intravitreally injected with AAV-DIO-EGFP. Due to AAV-DIO plasmid self-recombination during DNA amplification and viral vector production, flipped AAV-DIO-EGFP vectors label very few original retinal ganglion cells (i) and their axons (j). k, Reprogramming strategy in Prokr2^{CreERT2} mice and diagrams of AAV expression vectors. Mice were intravitreally injected with AAVs on day 1(D1), and subsequently fed with tamoxifen (TM) to activate expression of genes delivered by the Cre-dependent AAV-DIO system on D3 to D7. Mice were sacrificed for analysis on D42 or later. l, Reprogramming efficiencies of transcription factor combinations. In control group, flipped AAV-DIO-EGFP label very few endogenous RGCs. Scale bars=40 μm in panel d, =800 μm in panel f, =200 μm in panel g, =100 μm in panel h, =1000 μm in panel i, and =100 μm in panel j.

FIG. 11. Calcium imaging and optogenetics analysis of RGCs. a, Diagram of the in vivo calcium imaging setup. b, Representative image of regenerated RGC terminals in the SC region. c, Histogram distribution of orientation selective index (OSI) of all responsive terminals recorded in 3 mice. d, Cumulative percentage plot of OSI for all terminal data presented in panel c. e, f, Representative EPSC of light-evoked postsynaptic AMPA receptor response (e) and postsynaptic action potential (f) in a SC neuron from C57B6/J mice whose RGCs were labeled with ChR2. Scale bar=10 μm.

FIG. 12. In vivo reprogramming after damaging original RGCs. Pvalb is expressed in some subtypes of RGCs, therefore, Pvalb^{CreERT2}; Rosa26-tdTomato mice are used to establish condition of intraocular pressure increase (IPI)-caused damage RGCs and their axons. a, Confocal image of optic nerve from Pvalb^{CreERT2}; Rosa26-tdTomato mice. Axons of Pvalb-tdTomato⁺ RGCs are intact. b, Confocal image of optic nerve from Pvalb^{CreERT2}; Rosa26-tdTomato mice seven days after intraocular pressure increase (IPI)-caused damage. All axons are damaged by IPI. c-e, Confocal images of flat-mount retina samples from Pvalb^{CreERT2} Rosa26-tdTomato mice. Pvalb-tdTomato marks RGCs and their axons in undamaged normal mice (c). Seven days after intraocular pressure increase (IPI)-caused damage, there is a dramatic loss of Pvalb-tdTomato⁺ RGCs in retina of mice (d). Ripasudil treatment slows down degeneration of Pvalb-tdTomato⁺ RGCs, but survived RGCs still do not have intact axons (e). f, Diagram of in vivo neuronal reprogramming in Lgr5^{EGFP-IRES-CreERT2}; Rosa26-tdTomato mice after damaging original RGCs by IPI. Lgr5^{EGFP-IRES-CreERT2}; Rosa26-tdTomato mice were first fed with tamoxifen (TM) five times (from day −11 (D-11) to day −7 (D-7)) to label Lgr5⁺ amacrine interneurons with the Rosa26-tdTomato reporter to assist identity tracing. One week later, RGCs and their axons of mice were damaged by increase of intraocular pressure on D1. Mice received intravitreal injection of AAVs expressing Cre-dependent transcription factors on D7, and were fed with tamoxifen (TM) from D9 to D13 to activate gene expression. Mice were sacrificed on D49 or later for analysis. To protect Lgr5⁺ amacrine interneurons and other retinal neurons, mice were treated with Ripasudil eye drop daily from D1 to D49. Scale bars=100 μm.

FIG. 13. Confocal images of flat-mount retina samples and optic nerves. a) Pavalbumin (PV) positive RGCs and their axons in flat-mount retina of PV-CreERT2; Rosa26-tdTomato mice. b) After intraocular pressure increase, many RGCs die and their axons degenerate. c) Expression of Atoh7+Brn3B+Sox4 in survived RGCs causes these cells to regrow/regenerate axons. d) PV-positive RGC axons in optic nerve of PV-CreERT2; Rosa26-tdTomato mice. e) After intraocular pressure increase-caused RGC damage, axons of RGCs degenerate within the optic nerve. F) Regenerated RGC axons within the optic nerve after over expression of Atoh7+Brn3B+Sox4 in survived RGCs.

FIG. 14. Projection of regenerated RGC axons into brain visual areas. a) Regenerated RGC axons in the dorsal and ventral lateral geniculate nuclei. b) Regenerated RGC axons in the superior colliculus nucleus.

### DETAILED DESCRIPTION

### Definitions

Unless defined otherwise, all technical and scientific terms used herein have the same meaning as is commonly understood by one of ordinary skill in the art. As used herein, the below terms have the following meanings unless specified otherwise. Any methods, devices and materials similar or equivalent to those described herein can be used in the practice of the compositions and methods described herein. The following definitions are provided to facilitate understanding of certain terms used frequently herein and are not meant to limit the scope of the present disclosure. All references referred to herein are incorporated by reference in their entirety.

As used herein, the term “comprising” is intended to mean that the compositions and methods include the recited elements, but not excluding others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of other ingredients and substantial method steps recited. Embodiments defined by each of these transition terms are within the scope of this invention.

The term “about” means within ±10%, ±5% or ±1% of a given value or range. In one embodiment, about means ±10% of a given value or range. In another embodiment, about means ±5% of a given value or range. In another embodiment, about means ±1% of a given value or range.

“Expression control sequence” refers to a nucleic acid sequence that regulates the expression of a nucleotide sequence to which it is operably linked. An expression control sequence is “operably linked” to a nucleotide sequence when the expression control sequence controls and regulates the transcription and/or the translation of the nucleotide sequence. Thus, an expression control sequence can include promoters, enhancers, internal ribosome entry sites (IRES), transcription terminators, a start codon in front of a protein-encoding gene, splicing signals for introns, and stop codons. The term “expression control sequence” is intended to include, at a minimum, a sequence whose presence are designed to influence expression, and can also include additional advantageous components. For example, leader sequences and fusion partner sequences are expression control sequences. The term can also include the design of the nucleic acid sequence such that undesirable, potential initiation codons in and out of frame, are removed from the sequence. It can also include the design of the nucleic acid sequence such that undesirable potential splice sites are removed. It includes sequences or polyadenylation sequences (pA) which direct the addition of a polyA tail, i.e., a string of adenine residues at the 3′-end of a mRNA, which may be referred to as polyA sequences. It also can be designed to enhance mRNA stability. Expression control sequences which affect the transcription and translation stability, e.g., promoters, as well as sequences which effect the translation, e.g., Kozak sequences, suitable for use in insect cells are well known to those skilled in the art. Expression control sequences can be of such nature as to modulate the nucleotide sequence to which it is operably linked such that lower expression levels or higher expression levels are achieved.

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter, including e.g. attenuators or enhancers, but also silencers. A “constitutive” promoter is a promoter that is active in most tissues under most physiological and developmental conditions. An “inducible” promoter is a promoter that is physiologically or developmentally regulated, e.g. by the application of a chemical inducer. A “tissue specific” promoter is only active in specific types of tissues or cells.

A “vector” is a nucleic acid molecule (typically DNA or RNA) that serves to transfer a passenger nucleic acid sequence (i.e., DNA or RNA) into a host cell. Three common types of vectors include plasmids, phages and viruses. Preferably, the vector is a virus. Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be useful to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

A “viral vector” refers to a vector comprising some or all of the following: viral genes encoding a gene product, control sequences and viral packaging sequences. A “parvoviral vector” is defined as a recombinantly produced parvovirus or parvoviral particle that comprises a polynucleotide to be delivered into a host cell, either in vivo, ex vivo or in vitro. Examples of parvoviral vectors include e.g., adeno-associated virus vectors. Herein, a parvoviral vector construct refers to the polynucleotide comprising the viral genome or part thereof, and a transgene.

The term “administration” refers to introducing an agent into a patient. An effective amount can be administered, which can be determined by the treating physician or the like. The related terms and phrases “administering” and “administration of”, when used in connection with a compound or tablet (and grammatical equivalents) refer both to direct administration, which may be administration to a patient by a medical professional or by self-administration by the patient.

“Therapeutically effective amount” or “effective amount” refers to an amount of a drug or an agent that when administered locally via a pharmaceutical composition described herein to a patient suffering from a condition, will have an intended therapeutic effect, e.g., alleviation, amelioration, palliation or elimination of one or more symptoms of the condition in the patient. The full therapeutic effect does not necessarily occur immediately and may occur only after a therapeutically effective amount is being delivered continuously for a period of time. For slow release or controlled release formulation, “therapeutically effective amount” or “effective amount” may refer to the total amount that is effective over a period of time, which is slowly released from the delivery vehicle to the disease site at an ascertainable and controllable release rate that constantly provides an effective amount of the drug to the disease site. In some embodiments, “therapeutically effective amount” or “effective amount” refers to an amount released to the disease site at a given period of time, e.g., per day.

The term “pharmaceutically acceptable” refers to generally safe and non-toxic for human administration.

“Treatment”, “treating”, and “treat” are defined as acting upon a disease, disorder, or condition with an agent to reduce or ameliorate the harmful or any other undesired effects of the disease, disorder, or condition and/or its symptoms.

Regeneration of Retinal Ganglion Cells (RGCs) from Other Retinal Neuron Cells

Degeneration of retinal ganglion cells (RGCs) and their axons underlie vision loss in glaucoma and various optic neuropathies. There are currently no treatments available to restore lost vision in patients affected by these diseases. Regenerating RGCs and reconnecting the retina to the brain represent an ideal therapeutic strategy; however, mammals do not have a reservoir of retinal stem/progenitor cells poised to produce new neurons in adulthood.

It is demonstrated in the accompanying experimental examples RGCs can be regenerated by direct lineage reprogramming of retinal neurons. Amacrine and displaced amacrine interneurons were successfully converted into RGCs, which projected axons into brain retinorecipient areas. They conveyed visual information to the brain in response to visual stimulation, and were able to transmit electrical signals to postsynaptic neurons, in both normal animals and in an animal model of glaucoma where original RGCs have been damaged by elevated intraocular pressure.

In accordance with one embodiment of the present disclosure, therefore, provided is a method to reprogram a non-RGC neuron cell to become responsive to visual signals. The reprogramming, in one embodiment, entails activation (or increasing the biological activity) of one or more transcription factors in a non-RGC neural cell. In some embodiments, the transcription factor is a proneural transcription factor.

An example transcription factor is a POU-domain transcription factor, such as Brn3B. Brn3B (POU class 4 homeobox 2, or POU4F2, BRN3.2, or Brn-3b) is a member of the POU-domain transcription factor family and is involved in maintaining visual system neurons in the retina. A representative Brn3B gene of the human has a protein sequence of NP_004566.2 and an mRNA sequence of NM_004575.3. A representative Brn3B gene of the mouse has a protein sequence of NP_620394.2 and an mRNA sequence of NM_138944.3.

Another example transcription factor is a SOX (SRY-related HMG-box) transcription factor, such as Sox4. Sox4 (SRY-box transcription factor 4, or CSS10 or EVI16) is a member of the SOX (SRY-related HMG-box) transcription factor family and is involved in the regulation of embryonic development and in the determination of the cell fate. A representative Sox4 gene of the human has a protein sequence of NP_003098.1 and an mRNA sequence of NM_003107.3. A representative Sox4 gene of the mouse has a protein sequence of NP_033264.2 and an mRNA sequence of NM_009238.3.

Another example member of the SOX (SRY-related HMG-box) transcription factors family is Sox11. Sox11 (SRY-box transcription factor 11, or CSS9 or MRD27) is a member of the SOX (SRY-related HMG-box) transcription factor family and is involved in the regulation of embryonic development and in the determination of the cell fate. A representative Sox11 gene of the human has a protein sequence of NP_003099.1 and an mRNA sequence of NM_003108.4. A representative Sox11 gene of the mouse has a protein sequence of NP_033260.4 and an mRNA sequence of NM_009234.6.

Another example transcription factor is a basic helix-loop-helix transcription factor, such as Atoh7. Atoh7 (atonal bHLH transcription factor 7, or Math5, NCRNA, RNANC, PHPVAR, or bHLHa13) is a member of basic helix-loop-helix family of transcription factors and controls photoreceptor development. This gene plays a central role in retinal ganglion cell and optic nerve formation. A representative Atoh7 gene of the human has a protein sequence of NP_660161.1 and an mRNA sequence of NM_145178.4. A representative Atoh7 gene of the mouse has a protein sequence of NP_058560.1 or NP_001351577.1 and an mRNA sequence of NM_016864.3 or NM_001364648.2.

Another example transcription factor is a LIM/homeodomain transcription factor, such as Ils1. Ils1 (ISL LIM homeobox 1, or Isl-1 or ISLET1) is a member of LIM/homeodomain family of transcription factors and binds to the enhancer region of the insulin gene, among others, and may play an important role in regulating insulin gene expression. Ils1 is central to the development of pancreatic cell lineages and is required for motor neuron generation. A representative Ils1 gene of the human has a protein sequence of NP_002193.2 and an mRNA sequence of NM_002202.3. A representative Ils1 gene of the mouse has a protein sequence of NP_067434.3 and an mRNA sequence of NM_021459.4.

Example protein and nucleic acid sequences of these example transcription factors are provided in Table 1 below.

TABLE 1
Example Sequences

| Name | Sequence |
|---|---|
| Brn3B protein | >NP_004566.2 POU domain, class 4, transcription factor 2 [Homo sapiens] |
| (human) | MMMMSLNSKQAFSMPHGGSLHVEPKYSALHSTSPGSSAPIAPSASSPSSSSNAGGGGGGGGGGGGGGGRS |
| SEQ ID NO: 1 | SSSSSSGSSGGGGSEAMRRACLPTPPSNIFGGLDESLLARAEALAAVDIVSQSKSHHHHPPHHSPFKPDA |
| | TYHTMNTIPCTSAASSSSVPISHPSALAGTHHHHHHHHHHHHQPHQALEGELLEHLSPGLALGAMAGPDG |
| | AVVSTPAHAPHMATMNPMHQAALSMAHAHGLPSHMGCMSDVDADPRDLEAFAERFKQRRIKLGVTQADVG |
| | SALANLKIPGVGSLSQSTICRFESLTLSHNNMIALKPILQAWLEEAEKSHREKLTKPELFNGAEKKRKRT |
| | SIAAPEKRSLEAYFAIQPRPSSEKIAAIAEKLDLKKNVVRVWFCNQRQKQKRMKYSAGI |
| Brn3B coding | >NM_004575.3:249-1478 Homo sapiens POU class 4 homeobox 2 (POU4F2), |
| seq (human) | mRNA |
| SEQ ID NO: 2 | ATGATGATGATGTCCCTGAACAGCAAGCAGGCGTTTAGCATGCCGCACGGCGGCAGCCTGCACGTGGAGC |
| | CCAAGTACTCGGCACTGCACAGCACCTCGCCGGGCTCCTCGGCTCCCATCGCGCCCTCGGCCAGCTCCCC |
| | CAGCAGCTCGAGCAACGCTGGTGGTGGCGGCGGCGGCGGCGGCGGCGGCGGCGGCGGCGGAGGCCGAAGC |
| | AGCAGCTCCAGCAGCAGTGGCAGCAGCGGCGGCGGGGGCTCGGAGGCTATGCGGAGAGCCTGTCTTCCAA |
| | CCCCACCGAGCAATATATTCGGCGGGCTGGATGAGAGTCTGCTGGCCCGCGCCGAGGCTCTGGCAGCCGT |
| | GGACATCGTCTCCCAGAGCAAGAGCCACCACCACCATCCACCCCACCACAGCCCCTTCAAACCGGACGCC |
| | ACCTACCACACTATGAATACCATCCCGTGCACGTCGGCCGCCTCTTCTTCATCGGTGCCCATCTCGCACC |
| | CTTCCGCGTTGGCGGGCACGCACCACCACCACCACCATCACCACCACCACCACCACCAACCGCACCAGGC |
| | GCTGGAGGGCGAGCTGCTGGAGCACCTGAGTCCCGGGCTGGCCCTGGGCGCTATGGCGGGCCCCGACGGC |
| | GCTGTGGTGTCCACGCCGGCTCACGCGCCGCACATGGCCACCATGAACCCCATGCACCAAGCAGCGCTCA |
| | GCATGGCCCACGCGCACGGGCTGCCGTCGCACATGGGCTGCATGAGCGACGTGGACGCCGACCCGCGGGA |
| | CCTGGAGGCATTCGCCGAGCGCTTCAAGCAGCGACGCATCAAGCTGGGGGTGACCCAGGCAGATGTGGGC |
| | TCCGCGCTGGCCAACCTCAAGATCCCCGGCGTGGGCTCGCTTAGCCAGAGCACCATCTGCAGGTTCGAGT |
| | CCCTCACACTGTCCCACAATAATATGATCGCGCTCAAACCCATCCTGCAGGCATGGCTCGAGGAGGCCGA |
| | GAAGTCCCACCGCGAGAAGCTCACCAAGCCTGAACTCTTCAATGGCGCGGAGAAGAAGCGCAAGCGCACG |
| | TCCATCGCTGCGCCAGAGAAGCGCTCGCTCGAAGCCTACTTTGCCATTCAGCCTCGGCCCTCCTCTGAAA |
| | AGATCGCCGCCATCGCGGAGAAGCTGGACCTGAAGAAAAACGTGGTGCGCGTCTGGTTCTGCAACCAGAG |
| | GCAGAAACAGAAAAGAATGAAATATTCCGCCGGCATTTAG |
| Brn3B protein | >NP_620394.2 POU domain, class 4, transcription factor 2 [Mus musculus] |
| (mouse) | MMMMSLNSKQAFSMPHAGSLHVEPKYSALHSASPGSSAPAAPSASSPSSSSNAGGGGGGGGGGGGGGRSS |
| SEQID NO: 3 | SSSSSGSGGSGGGGGSEAMRRACLPTPPSNIFGGLDESLLARAEALAAVDIVSQSKSHHHHPPHHSPFKP |
| | DATYHTMNTIPCTSAASSSSVPISHPSALAGTHHHHHHHHHHHHQPHQALEGELLEHLSPGLALGAMAGP |
| | DGTWSTPAHAPHMATMNPMHQAALSMAHAHGLPSHMGCMSDVDADPRDLEAFAERFKQRRIKLGVTQAD |
| | VGSALANLKIPGVGSLSQSTICRFESLTLSHNNMIALKPILQAWLEEAEKSHREKLTKPELFNGAEKKRK |
| | RTSIAAPEKRSLEAYFAIQPRPSSEKIAAIAEKLDLKKNVVRVWFCNQRQKQKRMKYSAGI |
| Brn3B coding | >NM_138944.3:244-1479 Mus musculus POU domain, class 4, transcription |
| seq (mouse) | factor 2 (Pou4f2), mRNA |
| SEQ ID NO: 4 | ATGATGATGATGTCCCTGAACAGCAAGCAGGCGTTCAGCATGCCTCACGCAGGCAGCCTGCACGTGGAGC |
| | CCAAGTACTCGGCGCTACACAGTGCCTCCCCGGGCTCCTCTGCGCCCGCGGCGCCCTCGGCCAGTTCCCC |
| | TAGCAGCTCCAGCAACGCTGGCGGCGGCGGCGGTGGCGGCGGAGGCGGAGGCGGCGGCGGCCGGAGCAGC |
| | AGTTCCAGCAGCAGTGGCAGCGGCGGCAGCGGCGGCGGCGGGGGCTCGGAGGCGATGCGGAGAGCTTGTC |
| | TTCCAACCCCACCGAGCAATATATTCGGCGGGCTGGATGAGAGTCTGCTGGCCCGTGCCGAGGCTCTGGC |
| | CGCCGTGGACATCGTCTCCCAGAGTAAGAGCCACCACCACCATCCGCCCCACCACAGCCCCTTCAAGCCG |
| | GACGCCACTTACCACACCATGAACACCATCCCGTGCACGTCGGCAGCCTCCTCTTCTTCTGTGCCCATCT |
| | CGCACCCGTCCGCTCTGGCTGGCACCCATCACCACCACCACCACCACCATCACCACCATCACCAGCCGCA |
| | CCAGGCGCTGGAGGGCGAGCTGCTTGAGCACCTAAGCCCCGGGCTGGCCCTGGGAGCTATGGCGGGCCCC |
| | GACGGCACGGTGGTGTCCACTCCGGCTCACGCACCACACATGGCCACCATGAACCCCATGCACCAAGCAG |
| | CCCTGAGCATGGCCCACGCACATGGGCTGCCCTCGCACATGGGCTGCATGAGCGACGTGGATGCAGACCC |
| | GCGGGACCTGGAGGCGTTCGCCGAGCGTTTCAAGCAGCGACGCATCAAGCTGGGAGTGACCCAGGCAGAT |
| | GTGGGCTCGGCGCTGGCCAACCTCAAGATCCCGGGCGTGGGCTCGCTCAGCCAGAGCACCATCTGCAGGT |
| | TTGAGTCTCTCACGCTGTCACACAACAACATGATCGCGCTCAAGCCCATCCTGCAGGCGTGGCTGGAGGA |
| | AGCTGAGAAATCCCACCGCGAGAAGCTCACTAAGCCGGAGCTCTTCAATGGCGCGGAGAAGAAGCGCAAG |
| | CGCACGTCCATCGCGGCGCCGGAGAAGCGCTCTCTGGAAGCCTACTTCGCCATCCAGCCAAGGCCCTCCT |
| | CGGAGAAGATCGCGGCCATCGCCGAAAAGCTGGATCTCAAGAAAAATGTGGTGCGCGTCTGGTTCTGCAA |
| | CCAGAGGCAGAAACAGAAGAGAATGAAATACTCTGCCGGCATTTAG |
| Sox4 protein | >NP_003098.1 transcription factor SOX-4 [Homo sapiens] |
| (human) | MVQQTNNAENTEALLAGESSDSGAGLELGIASSPTPGSTASTGGKADDPSWCKTPSGHIKRPMNAFMVWS |
| SEQ ID NO: 5 | QIERRKIMEQSPDMHNAEISKRLGKRWKLLKDSDKIPFIREAERLRLKHMADYPDYKYRPRKKVKSGNAN |
| | SSSSAAASSKPGEKGDKVGGSGGGGHGGGGGGGSSNAGGGGGGASGGGANSKPAQKKSCGSKVAGGAGGG |
| | VSKPHAKLILAGGGGGGKAAAAAAASFAAEQAGAAALLPLGAAADHHSLYKARTPSASASASSAASASAA |
| | LAAPGKHLAEKKVKRVYLFGGLGTSSSPVGGVGAGADPSDPLGLYEEEGAGCSPDAPSLSGRSSAASSPA |
| | AGRSPADHRGYASLRAASPAPSSAPSHASSSASSHSSSSSSSGSSSSDDEFEDDLLDLNPSSNFESMSLG |
| | SFSSSSALDRDLDFNFEPGSGSHFEFPDYCTPEVSEMISGDWLESSISNLVFTY |
| Sox4 coding | >NM_003107.3:785-2209 Homo sapiens SRY-box transcription factor 4 |
| seq (human) | (SOX4), mRNA |
| SEQ ID NO: 6 | ATGGTGCAGCAAACCAACAATGCCGAGAACACGGAAGCGCTGCTGGCCGGCGAGAGCTCGGACTCGGGCG |
| | CCGGCCTCGAGCTGGGAATCGCCTCCTCCCCCACGCCCGGCTCCACCGCCTCCACGGGCGGCAAGGCCGA |
| | CGACCCGAGCTGGTGCAAGACCCCGAGTGGGCACATCAAGCGACCCATGAACGCCTTCATGGTGTGGTCG |
| | CAGATCGAGCGGCGCAAGATCATGGAGCAGTCGCCCGACATGCACAACGCCGAGATCTCCAAGCGGCTGG |
| | GCAAACGCTGGAAGCTGCTCAAAGACAGCGACAAGATCCCTTTCATTCGAGAGGCGGAGCGGCTGCGCCT |
| | CAAGCACATGGCTGACTACCCCGACTACAAGTACCGGCCCAGGAAGAAGGTGAAGTCCGGCAACGCCAAC |
| | TCCAGCTCCTCGGCCGCCGCCTCCTCCAAGCCGGGGGAGAAGGGAGACAAGGTCGGTGGCAGTGGCGGGG |
| | GCGGCCATGGGGGCGGCGGCGGCGGCGGGAGCAGCAACGCGGGGGGAGGAGGCGGCGGTGCGAGTGGCGG |
| | CGGCGCCAACTCCAAACCGGCGCAGAAAAAGAGCTGCGGCTCCAAAGTGGCGGGCGGCGCGGGCGGTGGG |
| | GTTAGCAAACCGCACGCCAAGCTCATCCTGGCAGGCGGCGGCGGCGGCGGGAAAGCAGCGGCTGCCGCCG |
| | CCGCCTCCTTCGCCGCCGAACAGGCGGGGGCCGCCGCCCTGCTGCCCCTGGGCGCCGCCGCCGACCACCA |
| | CTCGCTGTACAAGGCGCGGACTCCCAGCGCCTCGGCCTCCGCCTCCTCGGCAGCCTCGGCCTCCGCAGCG |
| | CTCGCGGCCCCGGGCAAGCACCTGGCGGAGAAGAAGGTGAAGCGCGTCTACCTGTTCGGCGGCCTGGGCA |
| | CGTCGTCGTCGCCCGTGGGCGGCGTGGGCGCGGGAGCCGACCCCAGCGACCCCCTGGGCCTGTACGAGGA |
| | GGAGGGCGCGGGCTGCTCGCCCGACGCGCCCAGCCTGAGCGGCCGCAGCAGCGCCGCCTCGTCCCCCGCC |
| | GCCGGCCGCTCGCCCGCCGACCACCGCGGCTACGCCAGCCTGCGCGCCGCCTCGCCCGCCCCGTCCAGCG |
| | CGCCCTCGCACGCGTCCTCCTCGGCCTCGTCCCACTCCTCCTCTTCCTCCTCCTCGGGCTCCTCGTCCTC |
| | CGACGACGAGTTCGAAGACGACCTGCTCGACCTGAACCCCAGCTCAAACTTTGAGAGCATGTCCCTGGGC |
| | AGCTTCAGTTCGTCGTCGGCGCTCGACCGGGACCTGGATTTTAACTTCGAGCCCGGCTCCGGCTCGCACT |
| | TCGAGTTCCCGGACTACTGCACGCCCGAGGTGAGCGAGATGATCTCGGGAGACTGGCTCGAGTCCAGCAT |
| | CTCCAACCTGGTTTTCACCTACTGA |
| Sox4 protein | >NP_033264.2 transcription factor SOX-4 [Mus musculus] |
| (mouse) | MVQQTNNAENTEALLAGESSDSGAGLELGIASSPTPGSTASTGGKADDPSWCKTPSGHIKRPMNAFMVWS |
| SEQ ID NO: 7 | QIERRKIMEQSPDMHNAEISKRLGKRWKLLKDSDKIPFIQEAERLRLKHMADYPDYKYRPRKKVKSGNAG |
| | AGSAATAKPGEKGDKVAGSSGHAGSSHAGGGAGGSSKPAPKKSCGPKVAGSSVGKPHAKLVPAGGSKAAA |
| | SFSPEQAALLPLGEPTAVYKVRTPSAATPAASSSPSSALATPAKHPADKKVKRVYLFGSLGASASPVGGL |
| | GASADPSDPLGLYEDGGPGCSPDGRSLSGRSSAASSPAASRSPADHRGYASLRAASPAPSSAPSHASSSL |
| | SSSSSSSSGSSSSDDEFEDDLLDLNPSSNFESMSLGSFSSSSALDRDLDFNFEPGSGSHFEFPDYCTPEV |
| | SEMISGDWLESSISNLVFTY |
| Sox4 coding | >NM_009238.3:679-2001 Mus musculus SRY (sex determining region Y)-box 4 |
| seq (mouse) | (Sox4), mRNA |
| SEQ ID NO: 8 | ATGGTACAACAGACCAACAACGCGGAGAACACTGAGGCTCTGCTGGCCGGGGAGAGCTCGGACTCGGGCG |
| | CCGGCCTGGAGCTGGGCATCGCGTCCTCCCCGACGCCTGGCTCCACCGCGTCGACGGGCGGCAAGGCGGA |
| | CGACCCCAGCTGGTGCAAGACGCCCAGTGGCCACATCAAGCGGCCCATGAACGCCTTTATGGTGTGGTCG |
| | CAGATCGAGCGGCGCAAGATCATGGAGCAGTCGCCCGACATGCACAACGCCGAGATCTCCAAGCGGCTAG |
| | GCAAACGCTGGAAGCTGCTCAAGGACAGCGACAAGATTCCGTTCATCCAGGAGGCGGAGCGGCTGCGCCT |
| | CAAGCACATGGCTGACTACCCTGACTACAAGTACCGGCCGCGAAAGAAGGTGAAGTCGGGCAACGCGGGC |
| | GCGGGATCGGCGGCCACAGCCAAGCCAGGGGAGAAGGGCGACAAGGTCGCGGGCAGCAGCGGCCACGCGG |
| | GAAGCAGCCACGCGGGGGGTGGCGCGGGCGGCAGCTCCAAGCCCGCGCCCAAGAAGAGCTGTGGCCCCAA |
| | GGTGGCGGGCAGCTCGGTCGGCAAGCCCCACGCTAAGCTGGTCCCGGCGGGCGGCAGCAAGGCGGCTGCA |
| | TCGTTCTCTCCAGAGCAAGCTGCCCTGCTGCCCCTGGGGGAGCCCACGGCCGTCTACAAGGTGCGGACTC |
| | CCAGTGCGGCCACTCCGGCCGCCTCCTCCTCGCCGTCCAGTGCGCTGGCCACCCCAGCCAAACACCCTGC |
| | CGACAAGAAAGTGAAGCGCGTCTACCTGTTTGGAAGCCTGGGCGCTTCGGCGTCTCCCGTCGGGGGCCTG |
| | GGAGCGAGCGCCGACCCCAGTGATCCACTGGGGTTGTACGAAGATGGAGGCCCGGGATGCTCGCCCGATG |
| | GCCGGAGTCTGAGCGGCCGCAGCAGCGCAGCATCATCGCCAGCCGCCAGCCGATCGCCCGCTGACCACCG |
| | CGGCTACGCCAGCCTACGCGCAGCCTCGCCCGCCCCGTCCAGCGCGCCCTCGCACGCGTCCTCCTCGCTC |
| | TCCTCGTCCTCTTCCTCCTCCTCGGGCTCTTCGTCGTCCGACGACGAGTTCGAAGACGACCTGCTCGACC |
| | TGAACCCCAGCTCAAACTTTGAGAGCATGTCCCTGGGCAGTTTCAGCTCCTCATCGGCGCTCGATCGGGA |
| | CCTGGATTTTAACTTCGAACCCGGCTCAGGCTCCCACTTCGAATTCCCGGACTATTGCACGCCCGAGGTG |
| | AGCGAGATGATCTCGGGAGATTGGCTGGAGTCCAGCATCTCTAACCTGGTCTTCACCTACTGA |
| Atoh7 protein | >NP_660161.1 protein atonal homolog 7 [Homo sapiens] |
| (human) | MKSCKPSGPPAGARVAPPCAGGTECAGTCAGAGRLESAARRRLAANARERRRMQGLNTAFDRLRRVVPQW |
| SEQ ID NO: 9 | GQDKKLSKYETLQMALSYIMALTRILAEAERFGSERDWVGLHCEHFGRDHYLPFPGAKLPGESELYSQRL |
| | FGFQPEPFQMAT |
| Atoh7 coding | >NM_145178.4:437-895 Homo sapiens atonal bHLH transcription factor 7 |
| seq (human) | (ATOH7), mRNA |
| SEQ ID NO: 10 | ATGAAGTCCTGCAAGCCCAGCGGCCCGCCGGCGGGAGCGCGCGTTGCACCCCCGTGCGCGGGCGGCACCG |
| | AGTGCGCGGGCACGTGCGCCGGGGCCGGGCGGCTGGAGAGCGCGGCGCGCAGGCGCCTGGCGGCCAACGC |
| | GCGCGAGCGCCGCCGCATGCAGGGGCTCAACACTGCCTTCGACCGCTTACGCAGGGTGGTTCCCCAGTGG |
| | GGCCAGGATAAAAAGCTGTCCAAGTACGAGACCCTGCAGATGGCCCTGAGCTACATCATGGCTCTGACCC |
| | GGATCCTGGCCGAGGCCGAGCGATTCGGCTCGGAGCGGGACTGGGTGGGTCTCCACTGTGAGCACTTCGG |
| | CCGCGACCACTACCTCCCGTTCCCGGGCGCGAAGCTGCCGGGCGAGAGCGAGCTGTACAGCCAGAGACTC |
| | TTCGGCTTCCAGCCCGAGCCCTTCCAGATGGCCACCTAG |
| Atoh7 protein | >NP_058560.1 protein atonal homolog 7 isoform l [Mus musculus] |
| (mouse | MKSACKPHGPPAGARGAPPCAGAAERAVSCAGPGRLESAARRRLAANARERRRMQGLNTAFDRLRRWPQ |
| isoform 1) | WGQDKKLSKYETLQMALSYIIALTRILAEAERDWVGLRCEQRGRDHPYLPFPGARLQVDPEPYGQRLFGF |
| SEQ ID NO: 11 | QPEPFPMAS |
| Atoh7 coding | >NM_016864.3:372-821 Mus musculus atonal bHLH transcription factor 7 |
| seq (mouse | (Atoh7), transcript variant 1, mRNA |
| isoform 1) | ATGAAGTCGGCCTGCAAACCCCACGGCCCTCCGGCGGGAGCTCGCGGCGCGCCCCCGTGCGCGGGCGCAG |
| SEQ ID NO: 12 | CCGAGCGCGCGGTCTCGTGCGCGGGGCCCGGGCGGCTGGAGAGCGCGGCGCGCAGGCGTCTGGCGGCCAA |
| | CGCGCGCGAGCGGCGCCGCATGCAGGGGCTGAACACGGCGTTCGACCGGCTGCGCAGGGTGGTGCCGCAG |
| | TGGGGCCAGGACAAGAAGCTGTCCAAGTACGAGACACTGCAGATGGCGCTCAGCTACATCATCGCGCTCA |
| | CCCGCATCCTAGCCGAAGCCGAGCGGGACTGGGTCGGGCTGCGCTGCGAGCAGCGGGGCCGCGATCACCC |
| | CTACCTCCCTTTCCCGGGTGCTAGGCTCCAGGTAGACCCTGAGCCCTATGGGCAGAGGCTCTTCGGCTTC |
| | CAGCCGGAGCCCTTCCCCATGGCCAGCTAA |
| Atoh7 protein | >NP_001351577.1 protein atonal homolog 7 isoform 2 [Mus musculus] |
| (mouse | MKSACKPHGPPAGARGAPPCAGAAERAVSCAGPGRLESAARRRLAANARERRRMQGLNTAFDRLRRVVPQ |
| isoform 2) | WGQDKKLSKYETLQMALSYIIALTRILAEAERDWVGLRCEQRGRDHPYLPFPGARLQVS |
| SEQ ID NO: 13 | |
| Atoh7 coding | >NM_001364648.2:372-761 Mus musculus atonal bHLH transcription factor 7 |
| seq (mouse | (Atoh7), transcript variant 2, mRNA |
| isoform 2) | ATGAAGTCGGCCTGCAAACCCCACGGCCCTCCGGCGGGAGCTCGCGGCGCGCCCCCGTGCGCGGGCGCAG |
| SEQ ID NO: 14 | CCGAGCGCGCGGTCTCGTGCGCGGGGCCCGGGCGGCTGGAGAGCGCGGCGCGCAGGCGTCTGGCGGCCAA |
| | CGCGCGCGAGCGGCGCCGCATGCAGGGGCTGAACACGGCGTTCGACCGGCTGCGCAGGGTGGTGCCGCAG |
| | TGGGGCCAGGACAAGAAGCTGTCCAAGTACGAGACACTGCAGATGGCGCTCAGCTACATCATCGCGCTCA |
| | CCCGCATCCTAGCCGAAGCCGAGCGGGACTGGGTCGGGCTGCGCTGCGAGCAGCGGGGCCGCGATCACCC |
| | CTACCTCCCTTTCCCGGGTGCTAGGCTCCAGGTTTCATGA |
| Sox11 protein | >NP_003099.1 transcription factor SOX-11 [Homo sapiens] |
| (human) | MVQQAESLEAESNLPREALDTEEGEFMACSPVALDESDPDWCKTASGHIKRPMNAFMVWSKIERRKIMEQ |
| SEQ ID NO: 15 | SPDMHNAEISKRLGKRWKMLKDSEKIPFIREAERLRLKHMADYPDYKYRPRKKPKMDPSAKPSASQSPEK |
| | SAAGGGGGSAGGGAGGAKTSKGSSKKCGKLKAPAAAGAKAGAGKAAQSGDYGGAGDDYVLGSLRVSGSGG |
| | GGAGKTVKCVFLDEDDDDDDDDDELQLQIKQEPDEEDEEPPHQQLLQPPGQQPSQLLRRYNVAKVPASPT |
| | LSSSAESPEGASLYDEVRAGATSGAGGGSRLYYSFKNITKQHPPPLAQPALSPASSRSVSTSSSSSSGSS |
| | SGSSGEDADDLMFDLSLNFSQSAHSASEQQLGGGAAAGNLSLSLVDKDLDSFSEGSLGSHFEFPDYCTPE |
| | LSEMIAGDWLEANFSDLVFTY |
| Sox11 coding | >NM_003108.4:339-1664 Homo sapiens SRY-box transcription factor 11 |
| seq (human) | (SOX11), mRNA |
| SEQ ID NO: 16 | ATGGTGCAGCAGGCGGAGAGCTTGGAAGCGGAGAGCAACCTGCCCCGGGAGGCGCTGGACACGGAGGAGG |
| | GCGAATTCATGGCTTGCAGCCCGGTGGCCCTGGACGAGAGCGACCCAGACTGGTGCAAGACGGCGTCGGG |
| | CCACATCAAGCGGCCGATGAACGCGTTCATGGTATGGTCCAAGATCGAACGCAGGAAGATCATGGAGCAG |
| | TCTCCGGACATGCACAACGCCGAGATCTCCAAGAGGCTGGGCAAGCGCTGGAAAATGCTGAAGGACAGCG |
| | AGAAGATCCCGTTCATCCGGGAGGCGGAGCGGCTGCGGCTCAAGCACATGGCCGACTACCCCGACTACAA |
| | GTACCGGCCCCGGAAAAAGCCCAAAATGGACCCCTCGGCCAAGCCCAGCGCCAGCCAGAGCCCAGAGAAG |
| | AGCGCGGCCGGCGGCGGCGGCGGGAGCGCGGGCGGAGGCGCGGGCGGTGCCAAGACCTCCAAGGGCTCCA |
| | GCAAGAAATGCGGCAAGCTCAAGGCCCCCGCGGCCGCGGGCGCCAAGGCGGGCGCGGGCAAGGCGGCCCA |
| | GTCCGGGGACTACGGGGGCGCGGGCGACGACTACGTGCTGGGCAGCCTGCGCGTGAGCGGCTCGGGCGGC |
| | GGCGGCGCGGGCAAGACGGTCAAGTGCGTGTTTCTGGATGAGGACGACGACGACGACGACGACGACGACG |
| | AGCTGCAGCTGCAGATCAAACAGGAGCCGGACGAGGAGGACGAGGAACCACCGCACCAGCAGCTCCTGCA |
| | GCCGCCGGGGCAGCAGCCGTCGCAGCTGCTGAGACGCTACAACGTCGCCAAAGTGCCCGCCAGCCCTACG |
| | CTGAGCAGCTCGGCGGAGTCCCCCGAGGGAGCGAGCCTCTACGACGAGGTGCGGGCCGGCGCGACCTCGG |
| | GCGCCGGGGGCGGCAGCCGCCTCTACTACAGCTTCAAGAACATCACCAAGCAGCACCCGCCGCCGCTCGC |
| | GCAGCCCGCGCTGTCGCCCGCGTCCTCGCGCTCGGTGTCCACCTCCTCGTCCAGCAGCAGCGGCAGCAGC |
| | AGCGGCAGCAGCGGCGAGGACGCCGACGACCTGATGTTCGACCTGAGCTTGAATTTCTCTCAAAGCGCGC |
| | ACAGCGCCAGCGAGCAGCAGCTGGGGGGCGGCGCGGCGGCCGGGAACCTGTCCCTGTCGCTGGTGGATAA |
| | GGATTTGGATTCGTTCAGCGAGGGCAGCCTGGGCTCCCACTTCGAGTTCCCCGACTACTGCACGCCGGAG |
| | CTGAGCGAGATGATCGCGGGGGACTGGCTGGAGGCGAACTTCTCCGACCTGGTGTTCACATATTGA |
| Sox11 protein | >NP_033260.4 transcription factor SOX-11 [Mus musculus] |
| (mouse) | MVQQAESSEAESNLPRDALDTEEGEFMACSPVALDESDPDWCKTASGHIKRPMNAFMVWSKIERRKIMEQ |
| SEQ ID NO: 17 | SPDMHNAEISKRLGKRWKMLKDSEKIPFIREAERLRLKHMADYPDYKYRPRKKPKTDPAAKPSAGQSPDK |
| | SAAGAKAAKGPGKKCAKLKAPAGKAGAGKAAQPGDCAAGKAAKCVFLDDDDEDDDEDDELQLRPKPDADD |
| | DDDEPAHSHLLPPPTQQQPPQLLRRYSVAKVPASPTLSSAAESPEGASLYDEVRAGGRLYYSFKNITKQQ |
| | PPPAPPALSPASSRCVSTSSSSGSSSGSGAEDADDLMFDLSLNFSQGAHSACEQPLGAGAAGNLSLSLVD |
| | KDLDSFSEGSLGSHFEFPDYCTPELSEMIAGDWLEANFSDLVFTY |
| Sox11 coding | >NM_009234.6:311-1498 Mus musculus SRY (sex determining region Y)-box |
| seq (mouse) | 11 (Sox11), mRNA |
| SEQ ID NO: 18 | ATGGTGCAGCAGGCCGAGAGCTCGGAAGCCGAGAGCAACCTGCCCCGGGACGCGCTGGACACCGAGGAGG |
| | GCGAGTTCATGGCGTGCAGCCCGGTGGCCCTGGACGAGAGCGACCCGGACTGGTGCAAGACGGCGTCGGG |
| | CCACATCAAACGGCCCATGAACGCCTTCATGGTGTGGTCCAAGATCGAGCGCAGGAAGATCATGGAGCAG |
| | TCGCCCGACATGCACAACGCCGAGATCTCCAAGAGGCTGGGCAAGCGCTGGAAGATGCTGAAGGACAGCG |
| | AGAAGATCCCGTTCATCAGGGAGGCGGAGCGCCTGCGCCTCAAGCACATGGCTGATTATCCCGACTACAA |
| | GTACCGGCCGCGCAAAAAGCCCAAGACGGACCCAGCGGCCAAGCCCAGCGCGGGCCAGAGCCCCGACAAG |
| | AGCGCGGCGGGCGCCAAGGCAGCCAAGGGCCCCGGCAAGAAGTGCGCCAAGCTCAAGGCGCCTGCGGGCA |
| | AGGCGGGCGCGGGCAAGGCGGCGCAGCCGGGGGACTGCGCCGCGGGCAAGGCAGCCAAGTGCGTCTTCCT |
| | GGACGACGACGATGAAGACGACGACGAAGATGACGAGCTGCAGCTACGGCCCAAGCCGGACGCTGACGAC |
| | GACGACGACGAGCCCGCGCACTCGCACCTGCTGCCGCCGCCGACGCAGCAGCAACCCCCTCAGCTGCTGA |
| | GGCGCTACAGCGTGGCCAAGGTCCCCGCCAGCCCCACGCTCAGCAGTGCCGCCGAGTCCCCCGAGGGCGC |
| | GAGCCTGTACGACGAAGTGCGCGCGGGCGGCCGGCTCTACTACAGCTTCAAGAACATCACCAAGCAGCAG |
| | CCTCCGCCCGCGCCTCCCGCGCTGTCGCCCGCGTCCTCCCGCTGCGTGTCCACCTCCTCATCCAGCGGCA |
| | GCAGCAGCGGCAGCGGCGCCGAGGATGCAGACGACCTCATGTTCGACCTGAGCTTGAATTTCTCCCAGGG |
| | CGCGCACAGCGCCTGCGAGCAGCCACTGGGCGCGGGAGCGGCGGGGAACCTGTCCCTGTCGCTGGTGGAT |
| | AAGGACCTGGATTCCTTCAGCGAGGGCAGCCTGGGTTCCCACTTCGAGTTCCCCGACTACTGCACGCCGG |
| | AGCTGAGCGAGATGATCGCGGGGGACTGGCTGGAGGCGAACTTCTCCGACCTGGTGTTCACGTATTGA |
| Ils1 protein | >NP_002193.2 insulin gene enhancer protein ISL-1 [Homo sapiens] |
| (human) | MGDMGDPPKKKRLISLCVGCGNQIHDQYILRVSPDLEWHAACLKCAECNQYLDESCTCFVRDGKTYCKRD |
| SEQ ID NO: 19 | YIRLYGIKCAKCSIGFSKNDFVMRARSKVYHIECFRCVACSRQLIPGDEFALREDGLFCRADHDWERAS |
| | LGAGDPLSPLHPARPLQMAAEPISARQPALRPHVHKQPEKTTRVRTVLNEKQLHTLRTCYAANPRPDALM |
| | KEQLVEMTGLSPRVIRVWFQNKRCKDKKRSIMMKQLQQQQPNDKTNIQGMTGTPMVAASPERHDGGLQAN |
| | PVEVQSYQPPWKVLSDFALQSDIDQPAFQQLVNFSEGGPGSNSTGSEVASMSSQLPDTPNSMVASPIEA |
| Ils1 coding | >NM_002202.3:225-1274 Homo sapiens ISL LIM homeobox 1 (ISL1), mRNA |
| seq (human) | ATGGGAGACATGGGAGATCCACCAAAAAAAAAACGTCTGATTTCCCTATGTGTTGGTTGCGGCAATCAGA |
| SEQ ID NO: 20 | TTCACGATCAGTATATTCTGAGGGTTTCTCCGGATTTGGAATGGCATGCGGCATGTTTGAAATGTGCGGA |
| | GTGTAATCAGTATTTGGACGAGAGCTGTACATGCTTTGTTAGGGATGGGAAAACCTACTGTAAAAGAGAT |
| | TATATCAGGTTGTACGGGATCAAATGCGCCAAGTGCAGCATCGGCTTCAGCAAGAACGACTTCGTGATGC |
| | GTGCCCGCTCCAAGGTGTATCACATCGAGTGTTTCCGCTGTGTGGCCTGCAGCCGCCAGCTCATCCCTGG |
| | GGACGAATTTGCGCTTCGGGAGGACGGTCTCTTCTGCCGAGCAGACCACGATGTGGTGGAGAGGGCCAGT |
| | CTAGGCGCTGGCGACCCGCTCAGTCCCCTGCATCCAGCGCGGCCACTGCAAATGGCAGCGGAGCCCATCT |
| | CCGCCAGGCAGCCAGCCCTGCGGCCCCACGTCCACAAGCAGCCGGAGAAGACCACCCGCGTGCGGACTGT |
| | GCTGAACGAGAAGCAGCTGCACACCTTGCGGACCTGCTACGCCGCAAACCCGCGGCCAGATGCGCTCATG |
| | AAGGAGCAACTGGTAGAGATGACGGGCCTCAGTCCCCGTGTGATCCGGGTCTGGTTTCAAAACAAGCGGT |
| | GCAAGGACAAGAAGCGAAGCATCATGATGAAGCAACTCCAGCAGCAGCAGCCCAATGACAAAACTAATAT |
| | CCAGGGGATGACAGGAACTCCCATGGTGGCTGCCAGTCCAGAGAGACACGACGGTGGCTTACAGGCTAAC |
| | CCAGTGGAAGTACAAAGTTACCAGCCACCTTGGAAAGTACTGAGCGACTTCGCCTTGCAGAGTGACATAG |
| | ATCAGCCTGCTTTTCAGCAACTGGTCAATTTTTCAGAAGGAGGACCGGGCTCTAATTCCACTGGCAGTGA |
| | AGTAGCATCAATGTCCTCTCAACTTCCAGATACACCTAACAGCATGGTAGCCAGTCCTATTGAGGCATGA |
| Ils1 protein | >NP_067434.3 insulin gene enhancer protein ISL-1 [Mus musculus] |
| (mouse) | MGDMGDPPKKKRLISLCVGCGNQIHDQYILRVSPDLEWHAACLKCAECNQYLDESCTCFVRDGKTYCKRD |
| SEQ ID NO: 21 | YIRLYGIKCAKCSIGFSKNDFVMRARSKVYHIECFRCVACSRQLIPGDEFALREDGLFCRADHDVVERAS |
| | LGAGDPLSPLHPARPLQMAAEPISARQPALRPHVHKQPEKTTRVRTVLNEKQLHTLRTCYAANPRPDALM |
| | KEQLVEMTGLSPRVIRVWFQNKRCKDKKRSIMMKQLQQQQPNDKTNIQGMTGTPMVAASPERHDGGLQAN |
| | PVEVQSYQPPWKVLSDFALQSDIDQPAFQQLVNFSEGGPGSNSTGSEVASMSSQLPDTPNSMVASPIEA |
| Ils1 coding | >NM_021459.4:267-1316 Mus musculus ISL1 transcription factor, |
| seq (mouse) | LIM/homeodomain (Isll), mRNA |
| SEQ ID NO: 22 | ATGGGAGACATGGGCGATCCACCAAAAAAAAAACGTCTGATTTCCCTGTGTGTTGGTTGCGGCAATCAAA |
| | TTCACGACCAGTATATTCTGAGGGTTTCTCCGGATTTGGAGTGGCATGCAGCATGTTTGAAATGTGCGGA |
| | GTGTAATCAGTATTTGGACGAAAGCTGTACGTGCTTTGTTAGGGATGGGAAAACCTACTGTAAAAGAGAT |
| | TATATCAGGTTGTACGGGATCAAATGCGCCAAGTGCAGCATAGGCTTCAGCAAGAACGACTTCGTGATGC |
| | GTGCCCGCTCTAAGGTGTACCACATCGAGTGTTTCCGCTGTGTAGCCTGCAGCCGACAGCTCATCCCGGG |
| | AGACGAATTCGCCCTGCGGGAGGATGGGCTTTTCTGCCGTGCAGACCACGATGTGGTGGAGAGAGCCAGC |
| | CTGGGAGCTGGAGACCCTCTCAGTCCCTTGCATCCAGCGCGGCCTCTGCAAATGGCAGCCGAACCCATCT |
| | CGGCTAGGCAGCCAGCTCTGCGGCCGCACGTCCACAAGCAGCCGGAGAAGACCACCCGAGTGCGGACTGT |
| | GCTCAACGAGAAGCAGCTGCACACCTTGCGGACCTGCTATGCCGCCAACCCTCGGCCAGATGCGCTCATG |
| | AAGGAGCAACTAGTGGAGATGACGGGCCTCAGTCCCAGAGTCATCCGAGTGTGGTTTCAAAACAAGCGGT |
| | GCAAGGACAAGAAACGCAGCATCATGATGAAGCAGCTCCAGCAGCAGCAACCCAACGACAAAACTAATAT |
| | CCAGGGGATGACAGGAACTCCCATGGTGGCTGCTAGTCCGGAGAGACATGATGGTGGTTTACAGGCTAAC |
| | CCAGTAGAGGTGCAAAGTTACCAGCCGCCCTGGAAAGTACTGAGTGACTTCGCCTTGCAAAGCGACATAG |
| | ATCAGCCTGCTTTTCAGCAACTGGTCAATTTTTCAGAAGGAGGACCAGGCTCTAATTCTACTGGCAGTGA |
| | AGTAGCATCGATGTCCTCGCAGCTCCCAGATACACCCAACAGCATGGTAGCCAGTCCTATTGAGGCATGA |

Methods of increasing the biological activity of a gene are known in the art. Increased biological activity can be increased expression of the protein or increased function of the protein, or both.

In some embodiments, at least one of the transcription factors is activated in the cell. In one embodiment, the biological activity of Brn3B is increased. In one embodiment, the biological activity of Sox4 is increased. In one embodiment, the biological activity of Atoh7 is increased. In one embodiment, the biological activity of Sox11 is increased. In one embodiment, the biological activity of Ils1 is increased.

In some embodiments, the biological activities of at least two of the transcription factors are increased. The two may be Brn3B and Sox4, Brn3B and Atoh7, Brn3B and Sox11, Brn3B and Ils1, Sox4 and Atoh7, Sox4 and Sox11, Sox4 and Ils1, Atoh7 and Sox11, Atoh7 and Ils1, or Sox11 and Ils1.

In some embodiments, the biological activities of at least three of the transcription factors are increased. The three may be Brn3B, Sox4 and Atoh7, Brn3B, Sox4 and Sox11, or Brn3B, Sox4 and Ils1, without limitation. In some embodiments, the biological activities of at least four of the transcription factors are increased. In some embodiments, the biological activities of all five of the transcription factors are increased.

### Activation of Endogenous Transcription Factor

In one example, the expression of the corresponding endogenous gene is activated or enhanced. For instance, the human cytomegalovirus (CMV) enhancer/promoter (referred to as CMV) is a natural mammalian promoter with high transcriptional activity. The CMV enhancer is a strong enhancer in various mammalian cells, and has been widely used to drive ectopic expression of various genes in a wide range of mammalian cells, and to drive ectopic expression of exogenous genes in broad tissues in transgenic animals. In some examples, the transcriptional activity of the CMV enhancer can be further improved by changing the natural NF-κB binding sites into artificially selected NF-κB binding sequences with high binding affinity (Wang et al., Protein Expression and Purification 142:16-24, 2018). U.S. Pat. No. 10,329,595 also reports the generation of two improved CMV promoters (SEQ ID NO:26 and 27). Other useful gene promoters and enhancers are also known in the art.

In some embodiments, the promoter or enhancer is one that regulates the expression of a gene constantly expressed in a neuron. Example genes that are expressed in a neuron, such as an amacrine cell, include Pax6, Tcfap2b, Gad1, GlyT1, RBPMS, and Prox1. Another example gene is synapsin 1. Example promoters/enhancers are provided in Table 2.

TABLE 2
Example Promoters/Enhancers

| Name | Promoter Sequence |
|---|---|
| Human | AGTGCAAGTGGGTTTTAGGACCAGGATGAG |
| synapsin 1 | GCGGGGTGGGGGTGCCTACCTGACGACCGA |
| promoter | CCCCGACCCACTGGACAAGCACCCAACCCC |
| SEQ ID | CATTCCCCAAATTGCGCATCCCCTATCAGA |
| NO: 24 | GAGGGGGAGGGGAAACAGGATGCGGCGAGG |
| | CGCGTGCGCACTGCCAGCTTCAGCACCGCG |
| | GACAGTGCCTTCGCCCCCGCCTGGCGGCGC |
| | GCGCCACCGCCGCCTCAGCACTGAAGGCGC |
| | GCTGACGTCACTCGCCGGTCCCCCGCAAAC |
| | TCCCCTTCCCGGCCACCTTGGTCGCGTCCG |
| | CGCCGCCGCCGGCCCAGCCGGACCGCACCA |
| | CGCGAGGCGCGAGATAGGGGGGCACGGGCG |
| | CGACCATCTGCGCTGCGGCGCCGGCGACTC |
| | AGCGCTGCCTCAGTCTGCGGTGGGCAGCGG |
| | AGGAGTCGTGTCGTGCCTGAGAGCGCAG |
| Human | GTTGACATTGATTATTGACTAGTTATTAAT |
| CMV-major | AGTAATCAATTACGGGGTCATTAGTTCATA |
| immediate- | GCCCATATATGGAGTTCCGCGTTACATAAC |
| early | TTACGGTAAATGGCCCGCCTGGCTGACCGC |
| promoter | CCAACGACCCCCGCCCATTGACGTCAATAA |
| SEQ ID | TGACGTATGTTCCCATAGTAACGCCAATAG |
| NO: 25 | GGACTTTCCATTGACGTCAATGGGTGGAGT |
| | ATTTACGGTAAACTGCCCACTTGGCAGTAC |
| | ATCAAGTGTATCATATGCCAAGTACGCCCC |
| | CTATTGACGTCAATGACGGTAAATGGCCCG |
| | CCTGGCATTATGCCCAGTACATGACCTTAT |
| | GGGACTTTCCTACTTGGCAGTACATCTACG |
| | TATTAGTCATCGCTATTAGCATGGTGATGC |
| | GGTTTTGGCAGTACATCAATGGGCGTGGAT |
| | AGCGGTTTGACTCACGGGGATTTCCAAGTC |
| | TCCACCCCATTGACGTCAATGGGAGTTTGT |
| | TTTGGCACCAAAATCAACGGGACTTTCCAA |
| | AATGTCGTAACAACTCCGCCCCATTGACGC |
| | AAATGGGCGGTAGGCGTGTACGGTGGGAGG |
| | TCTATATAAGCAGAGCTCCGTTTAGTGAAC |
| | G |
| Human | GTTGACATTGATTATTGACTAGTTATTAAT |
| CMV-major | AGTAATCAATTACGGGGTCATTAGTTCATA |
| immediate- | GCCCATATATGGAGTTCCGCGTTACATAAC |
| early | TTACGGTAAATGGCCCGCCTGGCTGACCGC |
| promoter | CCAACGACCCCCGCCCATTGACGTCAATAA |
| variant 1 | TGACGTATGTTCCCATAGTAACGCCAATAG |
| SEQ ID | GGACTTTCCATTGACGTCAATGGGTGGAGT |
| NO: 26 | ATTTACGGTAAACTGCCCACTTGGCAGTAC |
| | ATCAAGTGTATCATATGCCAAGTACGCCCC |
| | CTATTGACGTCAATGACGGTAAATGGCCCG |
| | CCTGGCATTATGCCCAGTACATGACCTTAT |
| | GGGACTTTCCTACTTGGCAGTACATCTACG |
| | TATTAGTCATCGCTATTAGCATGGTGATGC |
| | GGTTTTGGCAGTACATCAATGGGCGTGGAT |
| | AGCGGTTTGACTCACGGGGATTTCCAAGTC |
| | TCCACCCCATTGACGTCAATGGGAGTTTGT |
| | TTTGGCACCAAAATCAACGGGACTTTCCAA |
| | AATGTCGTAACAACTCCGCCCCATTGACGC |
| | AAATGGGCGGTAGGCGTGTAGGGTGGGAGG |
| | TCTATATAAGCAGAGCTCCGTTTAGTGAAC |
| | G |
| Human | GTTGACATTGATTATTGACTAGTTATTAAT |
| CMV-major | AGTAATCAATTACGGGGTCATTAGTTCATA |
| immediate-e | GCCCATATATGGAGTTCCGCGTTACATAAC |
| arly | TTACGGTAAATGGCCCGCCTGGCTGACCGC |
| promoter | CCAACGACCCCCGCCCATTGACGTCAATAA |
| variant 2 | TGACGTATGTTCCCATAGTAACGCCAATAG |
| SEQ ID | GGACTTTCCATTGACGTCAATGGGTGGAGT |
| NO: 27 | ATTTACGGTAAACTGCCCACTTGGCAGTAC |
| | ATCAAGTGTATCATATGCCAAGTACGCCCC |
| | CTATTGACGTCAATGACGGTAAATGGCCCG |
| | CCTGGCATTATGCCCAGTACATGACCTTAT |
| | GGGACTTTCCTACTTGGCAGTACATCTACG |
| | TATTAGTCATCGCTATTAGCATGGTGATGC |
| | GGTTTTGGCAGTACATCAATGGGCGTGGAT |
| | AGGGGTTTGACTCACGGGGATTTCCAAGTC |
| | TCCACCCCATTGACGTCAATGGGAGTTTGT |
| | TTTGGCACCAAAATCAACGGGACTTTCCAA |
| | AATGTCGTAACAACTCCGCCCCATTGACGC |
| | AAATGGGCGGTAGGCGTGTACGGTGGGAGG |
| | TCTATATAAGCAGAGCTCCGTTTAGTGAAC |
| | G |

A gene expression promoter or enhancer can be introduced to the target gene by a conventional knock-in technology, or with a CRISPR method.

There are also an abundance of techniques for gene activation based on CRISPR. In one example, an inactive Cas protein (e.g., Cas9) is fused to appropriate transcriptional effector domains. Commonly used transcriptional activator domains include VP64, the p65 domain of NF-κB, the Epstein Barr virus R transactivator (Rta), and the activator domain for heat shock factor 1 (HSF1). In the endogenous context, multiple transcription factors and cofactors work in synchrony to stimulate gene transcription. Indeed, CRISPR tools that recruit multiple unique transcriptional activators to a promoter outperform those bearing a single transcriptional activator domain or redundant copies of the same effector. Targeting multiple sites on the same promoter also increases gene activation with CRISPR. One of the most effective CRISPR effectors is the CRISPR Synergistic Activation Mediator (SAM) complex, which recruits three unique transcriptional activator domains to the targeted gene promoter. In this system, one transcriptional activator VP64 (a multimeric form of VP16) is directly fused to dCas9.

In another example, a dCas9-p300 CRISPR Gene Activator system (Signa Aldrich, Hilton, Isaac B., et al. Nature Biotechnology (2015)) is based on a fusion of dCas9 to the catalytic histone acetyltransferase (HAT) core domain of the human E1A-associated protein p300. This approach activates genes at both proximal and distal locations relative the transcriptional start site (TSS).

### Introduction of Exogenous Transcription Factors

A more conventional technique to increase the biological activity (or expression) of a transcription factor is to introduce an exogenous sequence that encodes the transcription factor, or the transcription factor protein. A protein can be introduced into a cell by means of enclosing the protein in a vehicle, such as a liposome. Example protein sequences of the transcription factors are provided in Table 1.

A coding sequence, such as a cDNA or mRNA, can also be introduced into a target cell. Example coding sequences of the transcription factors are provided in Table 1. In some embodiments, a nucleic acid construct is prepared that includes coding sequences of one or more of these transcription factors. The coding sequence can be functionally connected to a suitable promoter or enhancer. In some embodiments, the promoter or enhancer is specific to the target cell, such as a retinal interneuron. Example promoters are provided in Table 2.

The construct may be plasmid, or preferably a viral vector. Suitable viral vectors includes lentiviral vectors and AAV vectors.

A “recombinant adeno-associated viral (AAV) vector” (or “rAAV vector”) herein refers to a vector comprising one or more polynucleotide sequences of interest, a gene product of interest, genes of interest or “transgenes” that are flanked by at least one parvoviral or AAV inverted terminal repeat sequences (ITRs). Such rAAV vectors can be replicated and packaged into infectious viral particles when present in an insect host cell that is expressing AAV rep and cap gene products (i.e., AAV Rep and Cap proteins). When an rAAV vector is incorporated into a larger nucleic acid construct (e.g., in a chromosome or in another vector such as a plasmid or baculovirus used for cloning or transfection), then the rAAV vector is typically referred to as a “pro-vector” which can be “rescued” by replication and encapsidation in the presence of AAV packaging functions and necessary helper functions. Preferably, a gene product of interest is flanked by AAV ITRs on either side. Any AAV ITR may be used in the constructs of the invention, including ITRs from AAV1, AAV2, AAV3, AAV4, AAV5, AAV6, AAV7, AAV8, AAV9, AAV10, AAV11 and/or AAV12.

An AAV gene therapy vector for use in the present technology may be produced either in mammalian cells or in insect cells. Both methods are described in the art. For example Grimm et al. (2003 Molecular Therapy 7(6):839-850) disclose a strategy to produce AAV vectors in a helper virus free and optically controllable manner, which is based on transfection of only two plasmids into 293T cells. They disclose a method for production of a hybrid AAV vector comprising AAV2 ITRs and AAV5 capsid proteins. Further information can also be found in Blits et al. (2010) (Journal of Neuroscience methods 185(2):257-263). The terms “hybrid” and “pseudotyped” are used interchangeably herein and are used to indicate vectors of which the Rep proteins, ITRs and/or capsid proteins are of different serotypes. For example, the ITRs and the Rep proteins are of AAV2 and the capsid proteins are of AAV5. The term “chimeric” is used herein to describe that a single gene, such as for example the capsid, is composed of at least two sequences derived from different serotypes.

AAV can for example be produced in mammalian cells according to the following method, but is not limited thereto: The vector genome contains the transgene expression cassette flanked by two inverted terminal repeats (ITRs) derived from AAV serotype 2. The total length of the viral vector genome may not exceed the wild type genome size of 4.7 kB in order to maintain efficient packaging efficiency. A single capsid is composed of 60 viral proteins of either, VP1 (62 kDa), VP2 (73 kDa), or VP3 (87 kDa), at a ratio of 1:1:10. The manufacturing process of AAV vectors is based upon Ca(PO4)2 transfection of two plasmids into human embryonic kidney production cells (HEK293) in roller bottles (850 cm2 surface area) followed by purification of the encapsidated vector genomes by filtration and chromatography techniques. The first plasmid is the viral vector plasmid and contains an expression construct which is flanked by AAV2 ITRs. The second plasmid is the packaging plasmid and encodes the AAV rep type 2 and cap type 5 genes of the desired serotype and adenovirus early helper genes E2A, VA, E4 (pDPS). The genome of the production cell line comprises the adenovirus E1 to provide helper functions. Following co-transfection with the two plasmids in Iscove's Modified Dulbecco's Medium (IMDM) containing 10% fetal calf serum (FCS), the cells are incubated for three days in serum-free Dulbecco's modified Eagle's medium (DMEM) to allow vector production to occur. Vector production in roller bottles on average results in yields of 3×10³ vector genomes per cell or 4×10″ vector genomes per roller bottle (quantified by qPCR). Subsequently, the cell culture is lysed by a buffer containing Triton-X-100 and cell debris removed by low speed centrifugation. The clarified bulk is purified by AVB Sepharose affinity chromatography and formulated into PBS/5% Sucrose by concentration and diafiltration using a 400 kDa hollow fiber module (for example from Spectrum Laboratories).

AAV ITR and Rep sequences that may be used in the present invention for the production of rAAV vectors in insect cells can be derived from the genome of any AAV serotype. Generally, the AAV serotypes have genomic sequences of significant homology at the amino acid and the nucleic acid levels. This provides an identical set of genetic functions to produce virions which are essentially physically and functionally equivalent. For the genomic sequence of the various AAV serotypes and an overview of the genomic similarities see e.g. GenBank Accession number U89790; GenBank Accession number J01901; GenBank Accession number AF043303; GenBank Accession number AF085716; Chiorini et al. (1997, J. Vir. 71: 6823-33); Srivastava et al. (1983, J. Vir. 45:555-64); Chiorini et al. (1999, J. Vir. 73:1309-1319); Rutledge et al. (1998, J. Vir. 72:309-319); and Wu et al. (2000, J. Vir. 74: 8635-47). rAAV serotypes 1, 2, 3, 4 and 5 are preferred source of AAV nucleotide sequences for use in the context of the present invention. Preferably the AAV ITR sequences for use in the context of the present invention are derived from AAV1, AAV2, and/or AAVS. More preferably, the ITR sequences for use in the present invention are AAV2 ITR. Likewise, the Rep (Rep78/68 and Rep52/40) coding sequences are preferably derived from AAV1, AAV2, and/or AAVS, more preferably AAV2.

AAV Rep and ITR sequences are particularly conserved among most serotypes. The Rep78 proteins of various AAV serotypes are e.g., more than 89% identical and the total nucleotide sequence identity at the genome level between AAV2, AAV3A, AAV3B, and AAV6 is around 82% (Bantel-Schaal et al., 1999, J. Virol., 73(2):939-947). Moreover, the Rep sequences and ITRs of many AAV serotypes are known to efficiently cross-complement (i.e., functionally substitute) corresponding sequences from other serotypes in production of AAV particles in mammalian cells. US2003148506 reports that AAV Rep and ITR sequences also efficiently cross-complement other AAV Rep and ITR sequences in insect cells.

The AAV VP proteins are known to determine the cellular tropicity of the AAV virion. The VP protein-encoding sequences are significantly less conserved than Rep proteins and genes among different AAV serotypes. The sequences coding for the viral proteins (VP) VP1, VP2, and VP3 capsid proteins for use in the context of the present invention are derived from AAVS. Most preferably, VP1, VP2 and VP3 are AAVS VP1, VP2 and VP3. Alternatively, VP1, VP2 and VP3 are wild-type AAVS sequences. The ability of Rep and ITR sequences to cross-complement corresponding sequences of other serotypes allows for the production of pseudotyped rAAV particles comprising the capsid proteins of one serotype and the ITR sequences of another AAV serotype. Such pseudotyped rAAV particles are a part of the present invention.

Each serotype of AAV may be more suitable for one or more particular tissues. For instance, AAV2, AAV3, AAV4, AAVS, AAV7 and AAV8 may be suitable for retina; AAV1, AAV2, AAV4, AAVS, AAV7 and AAV10 may be suitable for neurons; AAV2, AAV4, AAV8 and AAV9 may be suitable for the brain; AAV3, AAVS, AAV6, AAV9 and AAV10 may be suitable for the lung; AAV1, AAV6, AAV9 and AAV10 may be suitable for the heart; AAV2, AAV3 and AAV6-10 may be suitable for the liver; all of the serotypes except AAVS may be suitable for muscle tissues; AAV2 and AAV10 may be suitable for the kidney; and AAV1, AAV7 and AAV9 may be suitable for the pancreas.

In one embodiment, the AAV is of serotype AAV2. In one embodiment, the AAV is of serotype AAV3. In one embodiment, the AAV is of serotype AAV4. In one embodiment, the AAV is of serotype AAV5. In one embodiment, the AAV is of serotype AAV7. In one embodiment, the AAV is of serotype AAV8.

In some embodiments, the AAV vector is an AAV2.7m8 vector which is an engineered capsid with a 10-amino acid insertion in adeno-associated virus (AAV) surface variable region VIII (VR-VIII) resulting in the alteration of an antigenic region of AAV2 and the ability to efficiently transduce retina cells following intravitreal administration (Bennett et al., J Struct Biol, 2020 Feb. 1; 209(2):107433. doi: 10.1016/j.jsb.2019.107433. Epub 2019 Dec. 16). In some embodiments, the AAV vector is an AAV-DJ (type 2/type 8/type 9 chimera) engineered from shuffling eight different wild-type native viruses (Katada Y, et al., 2019. PeerJ 7:e6317). In some embodiments, the AAV vector is a AAV7m8 vector (Ramachandran et al., Hum Gene Ther. 2017 February; 28(2):154-167. doi: 10.1089/hum.2016.111. Epub 2016 Oct. 17).

### Target Cells

The reprogramming can be done with a non-RGC cell in the retina, such as any retinal neuron that is not a RGC. In some embodiments, such a retinal neuron is an interneuron cell. Example of interneuron cells are amacrine cells, bipolar cells and horizontal cells. In some embodiments, the non-RGC cell is a photoreceptor. Also, the non-RGC cell, in some embodiments, can be a Müller cell.

In some embodiments, the amacrine cell is a Lgr5⁺ amacrine cell. In some embodiments, the amacrine cell is a Prokr2⁺ displaced amacrine cell. In some embodiments, the amacrine cell is a Lgr5⁺ amacrine cell, and the biological activities (expressions) of both Brn3B and Sox4 are increased in the Lgr5⁺ amacrine cell. In some embodiments, the amacrine cell is a Prokr2⁺ displaced amacrine cell, and the biological activities (expressions) of both Brn3B and Sox4 are increased in the Prokr2⁺ displaced amacrine cell. In some embodiments, the amacrine cell is a Prokr2⁺ displaced amacrine cell, and the biological activities (expressions) of all of Brn3B, Sox4 and Atoh7 are increased in the Prokr2⁺ displaced amacrine cell.

The target cells can be reprogrammed in vitro or in vivo. In reprogrammed in vitro, the cells are converted into regenerated RGCs, which can be implanted into a subject in need thereof. When reprogrammed in vivo, the regenerated RGCs can replace damaged or degenerated RGCs, thereby treating vision impairment or blindness

### Rejuvenation of Retinal Ganglion Cells (RGCs)

In another surprising discovery, the instant inventors showed that activation of the transcription factors of the present disclosure was also effective in reactivating damaged RGCs (Example 2). The reactivated RGCs were able to regrow functional axons which projected into the optic nerve and connected with the brain.

Accordingly, another embodiment of the present disclosure provides a method for improving the function of a retinal ganglion cell (RGC). The RGC may be a degenerated, damaged, aged, or even a normal/healthy RGC for which improved function is desired. In some embodiments, the method entails increasing the biological activity, in the RGC, of one or more genes selected from the group consisting of Atoh7, Brn3B, Sox4, Sox11, and Ils1.

Methods of increasing the biological activity of a gene are known in the art. Increased biological activity can be increased expression of the protein or increased function of the protein, or both.

In some embodiments, at least one of the transcription factors is activated in the cell. In one embodiment, the biological activity of Brn3B is increased. In one embodiment, the biological activity of Sox4 is increased. In one embodiment, the biological activity of Atoh7 is increased. In one embodiment, the biological activity of Sox11 is increased. In one embodiment, the biological activity of Ils1 is increased.

In some embodiments, the biological activities of at least two of the transcription factors are increased. The two may be Brn3B and Sox4, Brn3B and Atoh7, Brn3B and Sox11, Brn3B and Ils1, Sox4 and Atoh7, Sox4 and Sox11, Sox4 and Ils1, Atoh7 and Sox11, Atoh7 and Ils1, or Sox11 and Ils 1.

In some embodiments, the biological activities of at least three of the transcription factors are increased. The three may be Brn3B, Sox4 and Atoh7, Brn3B, Sox4 and Sox11, or Brn3B, Sox4 and Ils1, without limitation. In some embodiments, the biological activities of at least four of the transcription factors are increased. In some embodiments, the biological activities of all five of the transcription factors are increased.

Example methods for activating endogenous transcription factors and introducing exogenous transcription factors are described in more details above. The methods may be in vitro, or in vivo.

### Compositions and Regenerated/Rejuvenated Cells

Agents, reagents and compositions are also provided, which can facilitate the implementation of the instantly disclosed technologies. Also provided, in some embodiments, is a RGC cell regenerated or rejuvenated by the present technologies.

One embodiment of the present disclosure provides a nucleic acid construct that can be introduced into a target cell for the desired reprogramming of the cell. In some embodiments, the nucleic acid construct includes coding sequences encoding any one, two, three, four or all of the transcription factors disclosed herein. In one embodiment, the nucleic acid construct includes the coding sequence for Brn3B. In one embodiment, the nucleic acid construct includes the coding sequence for Sox4. In one embodiment, the nucleic acid construct includes the coding sequence for Atoh7. In one embodiment, the nucleic acid construct includes the coding sequence for Sox11. In one embodiment, the nucleic acid construct includes the coding sequence for Ils1. Example protein and coding sequences of these transcription factors are provided in Table 1.

In one embodiment, the nucleic acid construct includes the coding sequences for at least two of the transcription factors, which may be Brn3B and Sox4, Brn3B and Atoh7, Brn3B and Sox11, Brn3B and Ils1, Sox4 and Atoh7, Sox4 and Sox11, Sox4 and Ils1, Atoh7 and Sox11, Atoh7 and Ils1, or Sox11 and Ils1. In some embodiments, the nucleic acid construct includes the coding sequences for at least three of the transcription factors, which may be Brn3B, Sox4 and Atoh7, Brn3B, Sox4 and Sox11, or Brn3B, Sox4 and Ils1, without limitation. In some embodiments, the nucleic acid construct includes the coding sequences for at least four of the transcription factors. In some embodiments, the nucleic acid construct includes the coding sequences for all five of the transcription factors.

In some embodiments, the nucleic acid construct includes a promoter or enhancer associated with each coding sequence. The promoter or enhancer is active in retinal interneuron cells. Non-limiting examples are promoters of Pax6, Tcfap2b, Gad1, GlyT1, RBPMS, and Prox1, or those provided in Table 2. In a particular example, the promoter is the synapsin 1 promoter.

In some examples, the nucleic acid construct includes an expression vector which may be a plasmid vector or viral vector, such as an AAV vector. The AAV may be selected from the group consisting of AAV1, AAV2, AAV3, AAV4, AAV5, AAV6, AAV7, AAV8, AAV9, AAV10, AAV11 and AAV12.

In one embodiment, the AAV is of serotype AAV2. In one embodiment, the AAV is of serotype AAV3. In one embodiment, the AAV is of serotype AAV4. In one embodiment, the AAV is of serotype AAV5. In one embodiment, the AAV is of serotype AAV7. In one embodiment, the AAV is of serotype AAV8.

In some embodiments, the AAV vector is an AAV2.7m8 vector which is an engineered capsid with a 10-amino acid insertion in adeno-associated virus (AAV) surface variable region VIII (VR-VIII) resulting in the alteration of an antigenic region of AAV2 and the ability to efficiently transduce retina cells following intravitreal administration (Bennett et al., J Struct Biol, 2020 Feb. 1; 209(2):107433. doi: 10.1016/j.jsb.2019.107433. Epub 2019 Dec. 16). In some embodiments, the AAV vector is an AAV-DJ (type 2/type 8/type 9 chimera) engineered from shuffling eight different wild-type native viruses (Katada Y, et al., 2019. PeerJ 7:e6317). In some embodiments, the AAV vector is a AAV7m8 vector (Ramachandran et al., Hum Gene Ther. 2017 February; 28(2):154-167. doi: 10.1089/hum.2016.111. Epub 2016 Oct. 17).

Cells that are transfected with the vectors, and cells reprogrammed or rejuvenated by the instant technologies are also provided. In one embodiment, a mammalian cell is provided that is responsive to visual signals. In one embodiment, the cell is prepared by increasing the biological activity of one or more genes disclosed herein in a retinal cell, such as a retinal interneuron cell, or a degenerated, damage, or aged RGC. The retinal cell, in another embodiment, is a Müller cell. In yet another embodiment, the retinal cell is a photoreceptor. In some embodiments, the reprogrammed cell is a regenerated retinal ganglion cell (RGC). In some embodiments, the reprogrammed cell is a rejuvenated retinal ganglion cell (RGC).

In some embodiments, the regenerated or rejuvenated RGCs can project axons into discrete subcortical brain regions. In some embodiments, the regenerated or rejuvenated RGCs can establish retina-brain connections. In some embodiments, the regenerated or rejuvenated RGCs can respond to visual stimulation and transmit electrical signals into the brain.

In some embodiments, the mammalian cell is an animal cell. In some embodiments, the mammalian cell is a human cell.

### Treatments and Uses

Loss of RGCs is a leading cause of blindness in a group of diseases broadly categorized as optic neuropathies, including glaucoma, hereditary optic neuropathies, and disorders caused by toxins, nutritional defects and trauma. The present technology, therefore, can be used to treat vision impairment or vision loss (blindness).

In some embodiments, the treatment or use entails administering to a patient (e.g., into the retina or pupil of the patient) an agent capable of increasing the biological activity of one or more genes disclosed herein, such as Brn3B, Sox4, Atoh7, Sox11, and Ils1. In some embodiments, the biological activities of at least two of the transcription factors are increased. The two may be Brn3B and Sox4, Brn3B and Atoh7, Brn3B and Sox11, Brn3B and Ils1, Sox4 and Atoh7, Sox4 and Sox11, Sox4 and Ils1, Atoh7 and Sox11, Atoh7 and Ils1, or Sox11 and Ils1. In some embodiments, the biological activities of at least three of the transcription factors are increased. The three may be Brn3B, Sox4 and Atoh7, Brn3B, Sox4 and Sox11, or Brn3B, Sox4 and Ils1, without limitation. In some embodiments, the biological activities of at least four of the transcription factors are increased. In some embodiments, the biological activities of all five of the transcription factors are increased.

Example agents have been discussed above, such as nucleic acid constructs that introduce a promoter or enhancer to one or more of the corresponding endogenous transcription factor (e.g., CRISPR systems), nucleic acid constructs that encode one or more of the transcription factors, and expressed proteins of the transcription factors.

The administration may be topical application, ophthalmological application, or intravitreal injection, without limitation.

In some embodiments, the agent is an AAV vector or pharmaceutical composition including the AAV vector. In some embodiments, the AAV vector or pharmaceutical composition administered may be from 1×10⁶ to 1×10²⁰ genome copy (gc)/kg, or from 1×10⁷ to 1×10²⁰, or from 1×10⁸ to 1×10²⁰, or from 1×10⁸ to 1×10¹⁹, or from 1×10⁹ to 1×10¹⁹, or from 1×10⁹ to 1×10¹⁸, or from 1×10¹⁰ to 1×10¹⁸, or from 1×10¹¹ to 1×10¹⁷, or from 1×10¹² to 1×10¹⁷, or 1×10¹³ to 1×10¹⁶, 2×10¹³ to 2×10¹⁵, 8×10¹³ to 6×10¹⁴ gc/kg body weight of the subject. It is to be noted that dosage values may vary with the severity of the condition to be alleviated. For any particular subject, specific dosage regimens may be adjusted over time according to the individual need and the professional judgement of the person administering or supervising the administration of the compositions. Dosage ranges set forth herein are exemplary only and do not limit the dosage ranges that may be selected by medical practitioners.

In some embodiment, the treatment entails implanting a reprogrammed retinal cell that is disclosed herein (e.g., a regenerated RPC) into the patient's eye, wherein the retain cell is reprogrammed in vitro.

### EXAMPLES

### Example 1

### Reprogramming of Retinal Interneuron Cells

This example shows that other retinal neurons can be used as an endogenous cellular source for retinal ganglion cells regeneration. By ectopic expression of transcription factors important for RGC differentiation, amacrine and displaced amacrine interneurons can be reprogrammed into RGCs. Regenerated RGCs project axons into discrete subcortical brain regions. They respond to visual stimulation and are able to transmit electrical signals into the brain, both under normal conditions and in an animal model of glaucoma, where the original RGCs have been damaged by increased intraocular pressure.

### Methods

Mice and husbandry. The Lgr5^{EGFP-IRES-CreERT2} knock-in mouse strain, the Pvalb^{CreERT2} knock-in mouse strain, and the Rosa26-tdTomato reporter mouse strain were obtained from the Jackson laboratory. Lgr5^{EGFP-IRES-CreERT2} mice and Pvalb^{CreERT2} mice were crossed with Rosa26-tdTomato mice to generate Lgr5^{EGFP-IRES-CreERT2}; Rosa26-tdTomato mice and Pvalb^{CreERT2}; Rosa26-tdTomato mice, respectively.

The Prokr2^{CreERT2} mouse strain was generated by homologous recombination using the CRISPR/Cas9 technology. Briefly, in vitro transcribed Cas9 mRNA, sgRNA and a donor vector plasmid were mixed and injected into the pronucleus of fertilized eggs from C57BL/6J mice. The donor vector plasmid was designed to insert the coding region of CreERT2 followed by a PolyA sequence into the ATG start codon of the Prokr2 locus. The injected zygotes were cultured until blastocyst stage by 3.5 days, and were subsequently transferred into uterus of pseudopregnant females. F0 mice with correct genome targeting were further crossed with C57BL/6J mice to generate F1 Prokr2^{CreERT2} mice. Prokr2^{CreERT2} mice were crossed with Rosa26-tdTomato mice to generate the Prokr2^{CreERT2} Rosa26-tdTomato mice. The DNA sequence around the Prokr2 translation start site is:

(SEQ ID NO: 23)
5′GCCCACCTGTAGCATCATCAACATGGGACCCCA
GAACAGAAACACTAGCTTTG 3′

The translation start site is in bold, and the target sequence of the sgRNA used is highlighted with underline. The donor vector plasmid contains a 5′ 4 kb-homology arm, the CreERT2-polyA cassette and a 3′ 4 kb-homology arm that was constructed with the In-Fusion cloning method.

All mice were housed in an animal facility with a 12-hour light/12-hour dark cycle. Animal experiments were conducted in both male and female mice of 8-12 months of age, and all animal experiment procedures were approved by the Animal Care and Use Committee at ShanghaiTech University.

Construction and production of AAV vectors. Coding sequences of mouse Atoh7, Brn3B, Sox4, Sox11, Ils1 and EGFP were sub-cloned into the CAG-driven Cre-dependent expression vector (Addgene #22222), replacing the original Arch-GFP sequence. To co-express a transcription factor and EGFP from a single AAV vector, a P2A fragment was placed between the two coding sequences.

For AAV viral particle production, HEK293T cells were transfected with the AAV transgene plasmid, pAAV7m8 serotype plasmid and the pHelper plasmid using PEI. Cells were collected 48-72 hours later. Viral particles were purified with Iodixanol density gradient centrifugation, and tittered by qPCR.

Intravitreal AAV injection. Mice were anesthetized by IP injection of a mixture of ketamine (80 mg/kg) and xylazine (8 mg/kg), and their pupils were dilated with a topical administration of Phenylepherine Hydrochloride ophthalmic solution (2.5%). After a brief topical anesthesia with 0.5% Proparacaine Hydrochloride eye drop, a cornea puncture was made to reduce intraocular pressure, and a 1.5 ul of AAV viral particles was injected into the vitreous space with a 34-gauge needle. For injections of AAV mixtures, each AAV was first diluted to 1×10¹² particles/ml before mixing.

Glaucoma model. Mouse RGCs were damaged using an intraocular pressure increase (IPI)-induced ischemia/reperfusion (I/R) model that mimics acute angle closure glaucoma in clinic. With minor modifications of a previously reported protocol, the ocular anterior chamber of mice was annulated with a needle, which is connected through a tube to an elevated saline (with 0.1% Heparin) reservoir. By elevating the height of the saline reservoir to 150 cm above the eye, the inner retinal blood flow was halted (ischemia). The needle was removed to install the circulation (reperfusion) 60 minutes later. This protocol causes degeneration of all RGC axons and death of other retinal neurons. To prevent other retinal neurons from apoptosis, a solution of Rock inhibitor Ripasudil hydrochloride dehydrate (0.4% in PBS) was administrated to the eye surface of mice once a day.

Immunohistochemistry and imaging. After being transcardially perfused with saline (0.9% NaCl in ddH2O) and subsequently 4% PFA, eyes, optic nerves and brains of mice were collected and post-fixed in 4% PFA for 24 hours. Eyes and brain tissues were placed in 30% sucrose for cyroprotection, and sectioned using a Microtome Cryostat at thickness of 10 and 30 μm, respectively Immuno-histochemical stainings were performed according to a standard protocol. The following antibodies were used: rabbit-anti-RBPMS (Abcam,1:400) to label RGCs, mouse-anti-Bm3a (Santa Cruz Biotechnology,1:200) to label RGCs, rabbit anti-SMI-32 (Abcam, 1:400) to label α-RGCs, rabbit anti-melanopsin (Abeam, 1:500) to label ipRGC, rabbit anti-CART (cocaine- and amphetamine-regulated transcript) (Phoenix Peptide,1:2500) to label ON-OFF DSGCs, mouse anti-PSD95 (Abcam, 1:400) to label postsynaptic cell membrane. For secondary detections, Alexa Fluor 647 donkey anti-rabbit (IFKine™,1:400), Alexa Fluor 647 donkey anti-mouse (IFKine™,1:400), or Alexa. Fluor 488 donkey anti-rabbit (Abcam, 1:400) were used. Immuno-stained tissue sections were imaged with a Zeiss LSM880 confocal microscope, a Nikon spinning disk (CSU Sora) confocal microscope or a STED SP8 microscope.

In vivo calcium imaging. For surgery, mice were anaesthetized with urethane (1.5 g/kg), and placed in a stereotaxic device with eyes covered with ophthalmic ointment. A custom titanium head-plate was bonded to the skull with black dental cement (Fe₃O₄ was added to block light), roughly centered on lambda, parallel to the long axis of the mouse. A 3-mm craniotomy was performed over the posteromedial SC and inferior colliculus, and a coverslip with 3 mm diameter was then gently pressed upon the dura and the craniotomy was sealed with black dental cement. A piece of black-out cloth was attached on the head-plate to avoid light contamination by the visual stimulation during functional two-photon imaging.

Visual stimuli were generated using the Matlab (Mathworks) function Psychtoolbox and displayed on a corrected 17′ LCD monitor (Dell, 1280 by 1024 pixels, 75 Hz refresh rate) positioned 15 cm from the contralateral eye. The stimuli were a full screen of sine-wave drifting gratings presented on a gray homogeneous background (spatial frequency: 0.05 cycles/°, temporal frequency: 2 Hz). The gratings were presented for 5 repeats with is duration and 1-2 sinterstimulus interval. The stimuli were drifted in 8 directions orthogonally to 4 orientations at regular intervals of 45°.

Two-photon imaging of fluorescence from axonal terminals was monitored with a customized LotosScan microscope (LotosScan, Suzhou Institute of Biomedical Engineering and Technology) and coupled with a mode-locked Ti:Sa laser (Chameleon VISION-S, Coherent). The excitation wavelength was fixed at 920 nm. Imaging was performed using a 40×, 0.8 NA objective (Nikon). The beam size was large enough to overfill the back aperture of the 40× objective. Images were acquired at a frame rate of 50 Hz (480×240 pixels, 0.225 μ/pixel).

Images were analyzed in Matlab (Mathworks) and ImageJ (National Institutes of Health). For correcting lateral motion in the imaging data, a rigid-body transformation based frame-by-frame alignment was applied by using Turboreg software (ImageJ plugin). Terminals were identified by hand on the basis of size, shape, and brightness. Individual terminal time courses were extracted by averaging pixel intensity values within terminal masks in each frame. If brain pulsation were evident during imaging, these data were not used. Neuropil signal was subtracted by using the method previously reported⁴⁰. After this correction, responses (Ft) to each stimulus presentation were normalized by response in the 0.2 simmediately before the stimulus onset (F0). For each stimulus, the mean change in fluorescence (ΔF/F) was calculated by averaging responses to all stimulus conditions and trials. Visually responsive cells were defined by ANOVA across blank and stimulus presentation periods (P<0.05).

Whole-cell patch clamp recording of Lgr5⁺ amacrine interneurons. Mice were dark-adapted for over 2 hours before being euthanized Dissection of the retina was then performed in artificial cerebrospinal fluid (ACSF) containing 126 mM NaCl, 1.25 mM NaH₂PO_{4, 2.5} mM KCl, 2 mM CaCl₂, 2 mM MgCl₂, 10 mM glucose and 26 mM NaHCO₃ under infrared light. Retinal slices were cut manually with razor blade and then were attached to a piece of filter paper, which is transferred to the recording chamber on the stage of microscope and perfused with oxygenated (95% O₂/5% CO₂) ACSF. Lgr5-tdTomato⁺ cells in INL were identified using two-photon microscope and targeted for whole-cell patch-clamp recording under infrared light. Pipettes (4-7 MΩ) were filled with intracellular solution containing 120 mM Cs-methanesulfonate, 5 mM NaCl, 10 mM HEPES, 5 mM EGTA, 5 mM QX314, 0.5 mM CaCl₂, 4 mM ATP, 0.5 mM GTP for voltage-clamp recordings or 123 mM K-gluconate, 10 mM KCl, 10 mM HEPES, 2 mM EGTA, 1 mM CaCl₂, 1 mM MgCl₂, 4 mM ATP, 0.5 mM GTP for current-clamp recordings. All reagents used above were from Sigma. Alexa488 hydradize (0.2 mM, ThermoFisher) was added in the intracellular solutions to visualize the morphology of the recorded cell. Signals were acquired and processed with a Multiclamp 700 A amplifier and the pClamp 10 software suite (Molecular Devices). Signals were filtered at 1 kHz and sampled at 10 kHz (Digidata 1440A, Molecular Devices). EPSCs were recorded at the reverse potential of Cl⁻ (−67 mV), and IPSCs were recorded at 0 mV. A white LED light controlled by the recording computer was used to deliver a full field light stimulation.

In vitro whole-cell patch clamp recording of SC neurons. Deeply anesthetized mice were transcardially perfused with an ice-cold oxygenated (95% O₂, 5% CO₂) cutting solution containing 92 mM Choline-chloride, 2.5 mM KCl, 1.2 mM NaH2PO4, 30 mM NaHCO3, 10 mM MgSO4, 0.5 mM CaCl2, 25 mM Glucose, 5 mM Na-ascorbate, 3 mM Sodium Pyruvate and 2 mM Thiourea. The pH value of the cutting solution was adjusted to 7.3-7.4 by adding concentrated HCl and the osmolarity was adjusted to 310-315 mOsm. After being removed from the skull, brain tissues containing the SC region were cut into 300 μm coronal slices within the cutting solution, using a vibrating blade microtome (VT1200 S, Leica Biosystems). Slices were then incubated in the same cutting solution at 31-32° C. for 15 minutes, before being transferred into a holding chamber containing room-temperature oxygenated holding solution (92 mM NaCl, 30 mM NaHCO3, 1.25 mM NaH2PO4, 2.5 mM KCl, 2 mM MgSO4, 2 mM CaCl₂ and 25 mM Glucose, 20 mM HEPES, 5 mM Na-ascorbate, 3 mM Sodium Pyruvate, and 2 mM Thiourea, with a pH value of 7.3-7.4 and a osmolarity value of 310-315 mOsm). After storing for one hour, the slices were transferred into a recording chamber containing room-temperature oxygenated recording solution (119 mM NaCl, 24 mM NaHCO3, 1.25 mM NaH2PO4, 2.5 mM KCl, 2 mM MgSO4, 2 mM CaCl₂ and 12.5 mM Glucose). Three to five slices containing the SC region were typically produced from one animal. Recordings were taken from brain slices containing the middle SC region.

Whole-cell patch clamp recordings of synaptic responses were made using a 2-4 MΩ glass pipettes with an internal solution of 125 mM K-gluconate,20 mM KCl, 0.5 mM EGTA, 10 mM HEPES-NaOH, 10 mM P-Creatine, 4 mM ATP-Mg, and 0.3 mM GTP (pH 7.3). Blue stimulation light was produced by a 470 nm LED (Thorlabs, 35 mW/mm²) and applied through an 40× objective (OLYMPUS). Stimulation duration at 5 ms was found to be able to saturate postsynaptic responses recorded. Neurons had input resistances in a range of 1-5 GΩ and series resistances less than 20 Ma Recordings were performed with the following protocol: The membrane potential was first held at −70 mV to record the light-evoked AMPA receptor-mediated synaptic currents (NMDA receptors were presumably blocked by magnesium at this holding potential). The membrane holding potential was then switched to +55 mV to record a mixture of AMPA and NMDA receptors-mediated currents. Under this condition, AMPA receptor antagonist CNQX (10 mM) was then added to the recording solution to block AMPA receptor-mediated synaptic currents, allowing detection of NMDA receptor-mediated EPSCs. Next, the recording was switched to current clamp mode to detect action potential. Applications of the AMPA receptor antagonist CNQX (Tocris) and the NMDA receptor antagonist D-APV (Tocris) were performed by adding respective drugs into the bathing recording solution. All recordings were made with an Axon700B amplifier and digitized using a Digidata1440 analog-to-digital board. Stimulation and data acquisition were performed with the pClamp software and digitized at 50 kHz. All equipment and software are from Axon Instruments/Molecular Devices (Molecular Devices, CA).

Statistics. Differences between two groups were compared using a two-tailed Student's t-test.

### Results

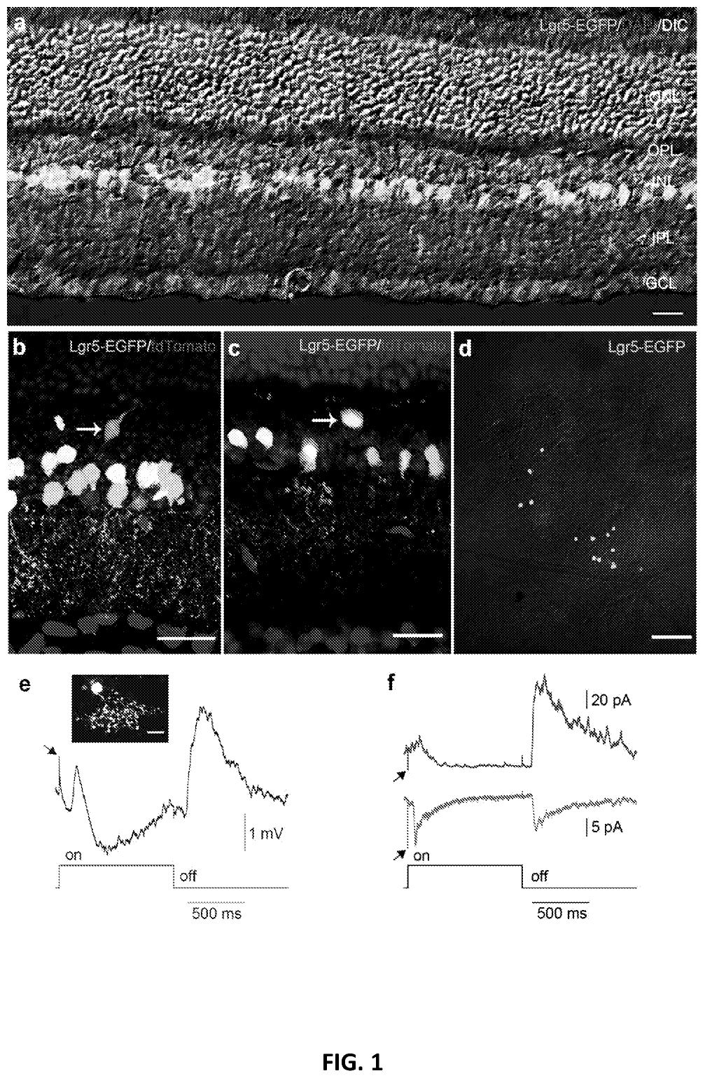

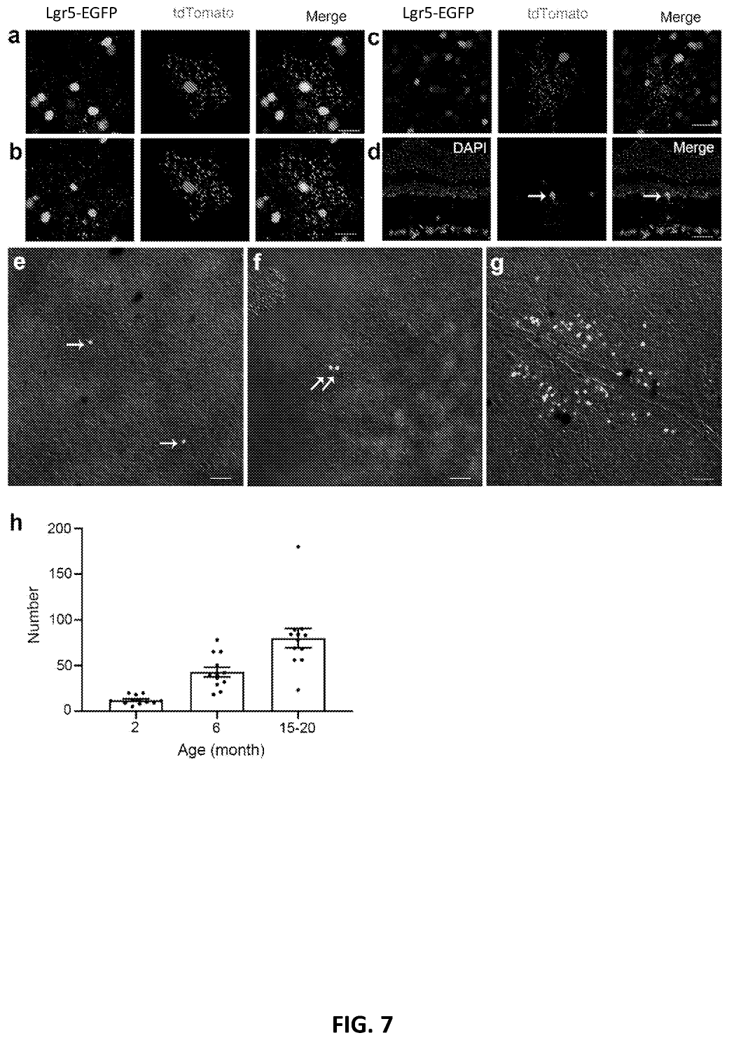

Reprogram Lgr5⁺ amacrine interneurons into RGCs in vivo. We first used the Lgr5^{EGFP-IRES-CreERT2}; Rosa26-tdTomato mouse strain to test whether RGCs could be regenerated from amacrine interneurons. Lgr5 is expressed in a subset of retinal cells located in the vitreous side of the inner nuclear layer (FIG. 1a). These Lgr5⁺ cells not only exhibit typical morphology of amacrine interneurons (FIG. 1a-c and 7a-d), but also have active synaptic connections in response to light stimulation (FIG. 1e, f). They could receive both excitatory and inhibitory postsynaptic currents (EPSCs and IPSCs) and be depolarized or hyperpolarized by them (FIG. 1e, f), as revealed by targeted patch-clamp recordings, suggesting that they are indeed mature amacrine interneurons. However, when Lgr5⁺ amacrine interneurons are labeled with the tdTomato reporter and lineage traced in adult mice, very few (less than one cell per retina at any given point of time) tdTomato⁺ bipolar cells and horizontal cells could be detected a few months later (FIG. 1b, c), indicating that some Lgr5⁺ amacrine cells can turn off Lgr5 expression and transdifferentiate into other retinal lineages, exhibiting limited regenerative potential. As mice age, a small number of Lgr5⁺ amacrine cells could be detected in the retinal ganglion cell layer, suggesting that they might be capable of migrating from the inner nuclear layer to the retinal ganglion layer (FIG. 1d and 7e-g). The number of Lgr5⁺ cells within the retinal ganglion cell layer increases with age (FIG. 7h), and some of these cells turn off Lgr5 expression, but they never turn into RGCs.

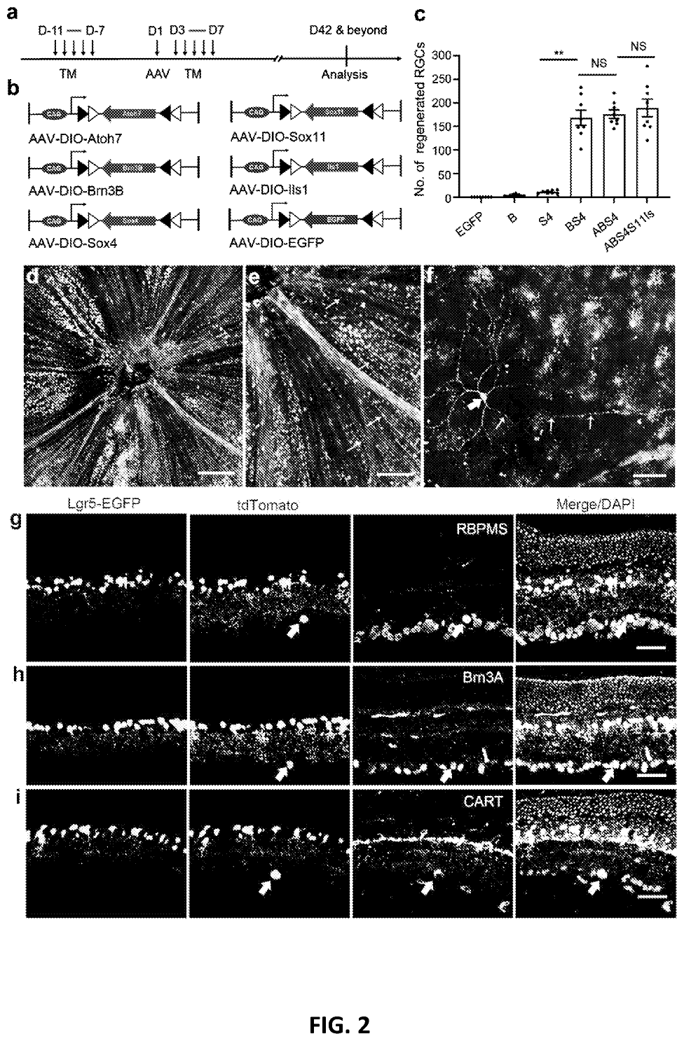

To investigate whether Lgr5⁺ amacrine interneurons could be reprogrammed into RGCs, we devised an in vivo lineage tracing and reprogramming strategy (FIG. 2a). We first labeled Lgr5⁺ amacrine neurons with the Rosa26-tdTomato reporter, and then ectopically expressed genes essential for RGC fate determination specifically in these cells, using the Cre-dependent double-floxed inverted open reading frame (DIO) expression system delivered via an adenovirus-associated virus (AAV) (FIG. 2b). We analyzed the generation of RGCs from Lgr5⁺ amacrine cells by examining the presence of tdTomato⁺ cells with RGC morphology in flat-mount retina samples, and the presence of tdTomato⁺ axons in optic nerves at later time points.

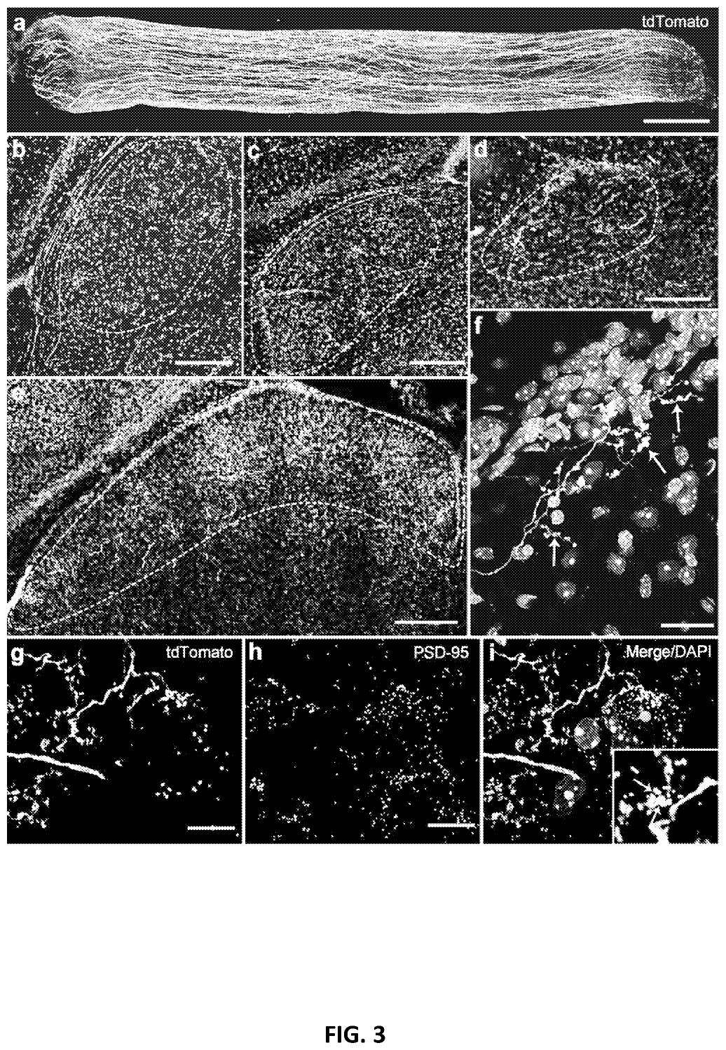

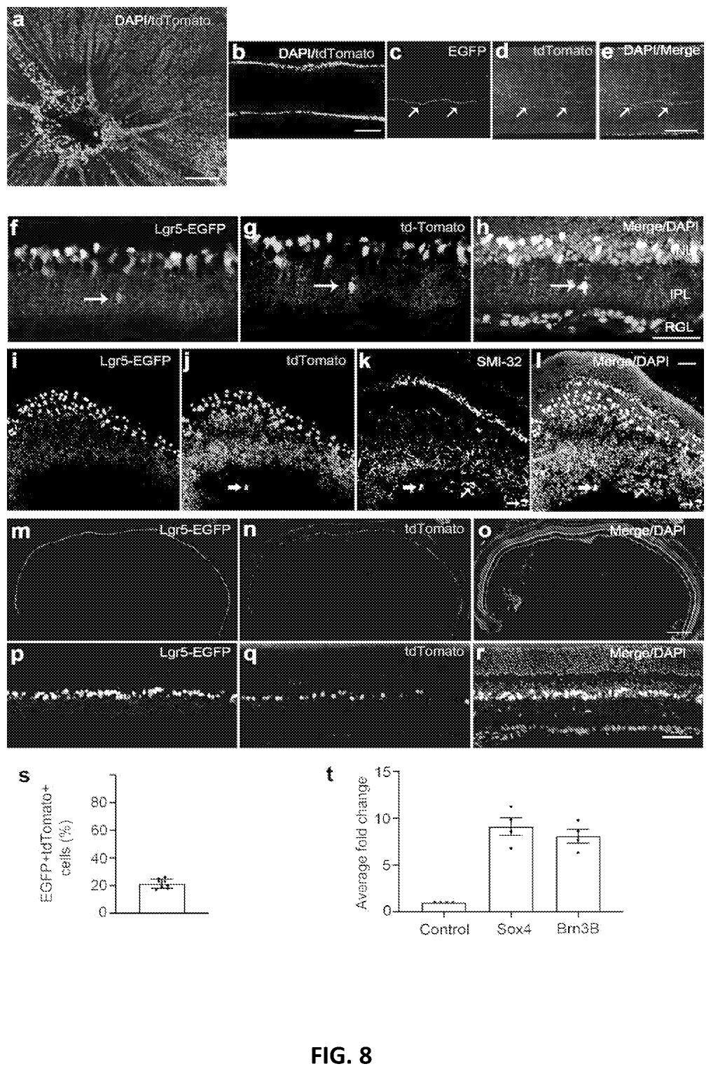

We did not observe any tdTomato⁺ RGC cells in flat-mount retina samples and tdTomato⁺ axons in optic nerves from control mice intravitreally injected with AAV-DIO-EGFP (FIGS. 2c and 8a-e). However, tdTomato⁺ cells with RGC morphology could be detected in retina samples from mice injected with AAVs, expressing a set of genes important for RGC fate determination (Atoh7, Brn3B, Sox4, Sox11 and Isl1). Six weeks after induced gene expression, tdTomato⁺ cells with RGC morphology were observed in retina samples (FIG. 2d-f). These cells projected axon-like projections towards the optic disc and extended into the optic nerve (FIG. 3a). Their cell bodies were located in the retinal ganglion cell layer, and can be stained with RGC-specific markers RBPMS and Brn3A (FIG. 2g, h). Therefore, these cells could be potentially considered as newly generated RGCs.

On average, about 180 new RGCs per retina were regenerated 6 weeks after viral injection (FIG. 2c). This number is much higher than that of Lgr5⁺ amacrine cells present in the retinal ganglion layer when in vivo reprogramming was initiated (about 10-15 cells in the retinal ganglion cell layer of 2-3 month-old mice) (FIG. 7h). This result suggests that ectopic expression of RGC fate-determining factors in Lgr5⁺ amacrine cells can trigger the migration of some of these cells from the inner nuclear layer to the ganglion cell layer. In support of this notion, tdTomato⁺ cells with lower Lgr5-EGFP expression level were detected in the inner plexiform layer (FIG. 8f-h).

We tested the reprogramming activities of single transcription factors and their combinations and found that, even single transcription factor (Brn3B or Sox4) was capable of reprogramming Lgr5⁺ amacrine interneurons into RGCs, but with very low efficiency (FIG. 2c). Combination of Brn3B and Sox4 dramatically synergized reprogramming activity. Addition of Atoh7 to the Brn3B+Sox4 combination did not further improve reprogramming efficiency much (FIG. 2c). Therefore, we used the Brn3B+Sox4 combination for the rest of experiments unless otherwise noted.

RGCs are a heterogeneous type of retina neurons that can be classified into distinct subtypes. We performed immuno-histological analysis with subtype-specific antibodies and found that, regenerated RGCs could be identified with anti-CART (for ON OFF directionally selective ganglion cells) and anti-SMI-32 (for α ganglion cells) (FIGS. 2i and 8i-l), but we did not detect any melanopsin-expressing intrinsically photosensitive ganglion cells. Together, these results suggest that ectopic expression of specific transcription factors is capable of reprogramming Lgr5⁺ amacrine interneurons into RGCs, and regenerated RGCs are subtype-specific.

Regenerated RGCs project axons into visual nuclei in the brain. To determine whether regenerated RGCs could rewire appropriately in the brain, we examined the axons of regenerated RGCs along the retinofugal pathway and their projections to the main brain retinorecipient areas. Six weeks after viral injection, many axons of regenerated RGC have traversed the entire optic nerve, passed the optic chiasm, and navigated into visual nuclei in the brain, including the dorsal and ventral lateral geniculate nucleus (dLGN and vLGN), the pretectal area, and the superior colliculus (SC) (FIG. 3). We did not observe aberrant projections of regenerated RGC axons to brain regions unrelated to the visual pathway. Within retinorecipient areas, micron-sized varicosities are observed along axonal arborizations of regenerated RGCs. These varicosities are in close apposition to staining for the postsynaptic density protein PSD-95 (FIG. 3g-i), suggesting that they are putative presynaptic boutons.

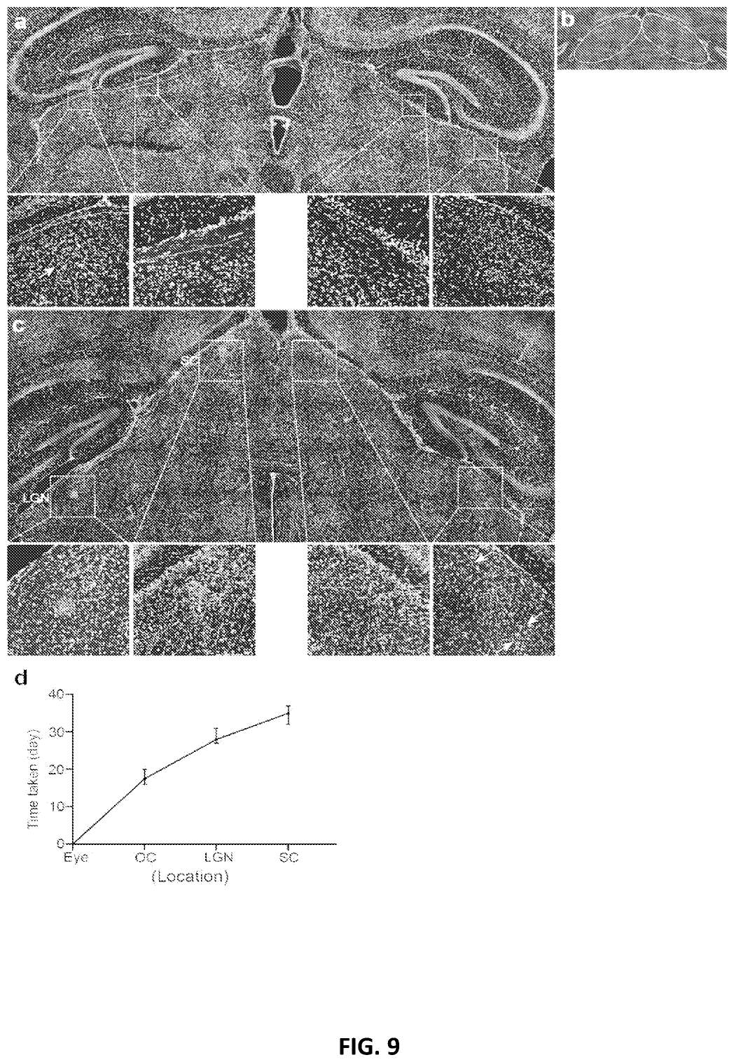

We determined the time course of axonal projection of regenerated RGCs to three important brain visual locations, the optic chiasma (OC), LGN and SC, by analyzing when regenerated RGC axons were first detected in these areas on brain slices after viral injection. We found that it took approximately 18 days for RGC axons to reach OC, 28 days to reach LGN and 35 days to reach the most distal visual target SC (FIG. 9). Together, these data demonstrate that Lgr5⁺ amacrine interneuron-derived RGCs are capable of projecting axons into appropriate brain areas, establishing retina-brain connection.

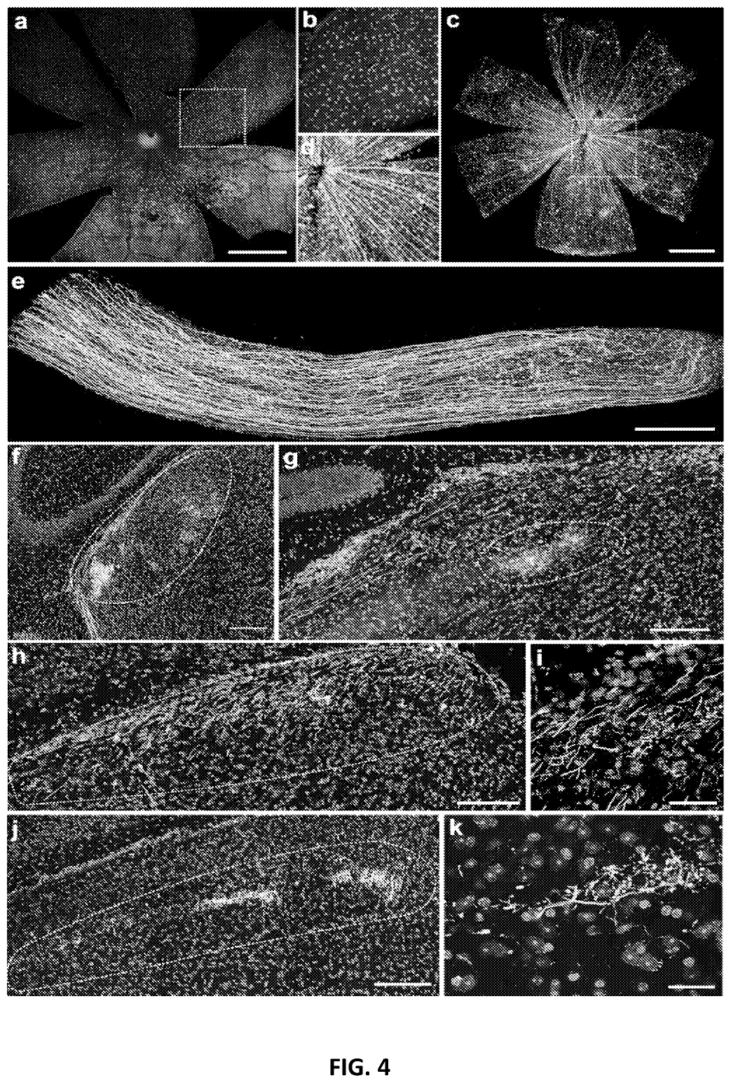

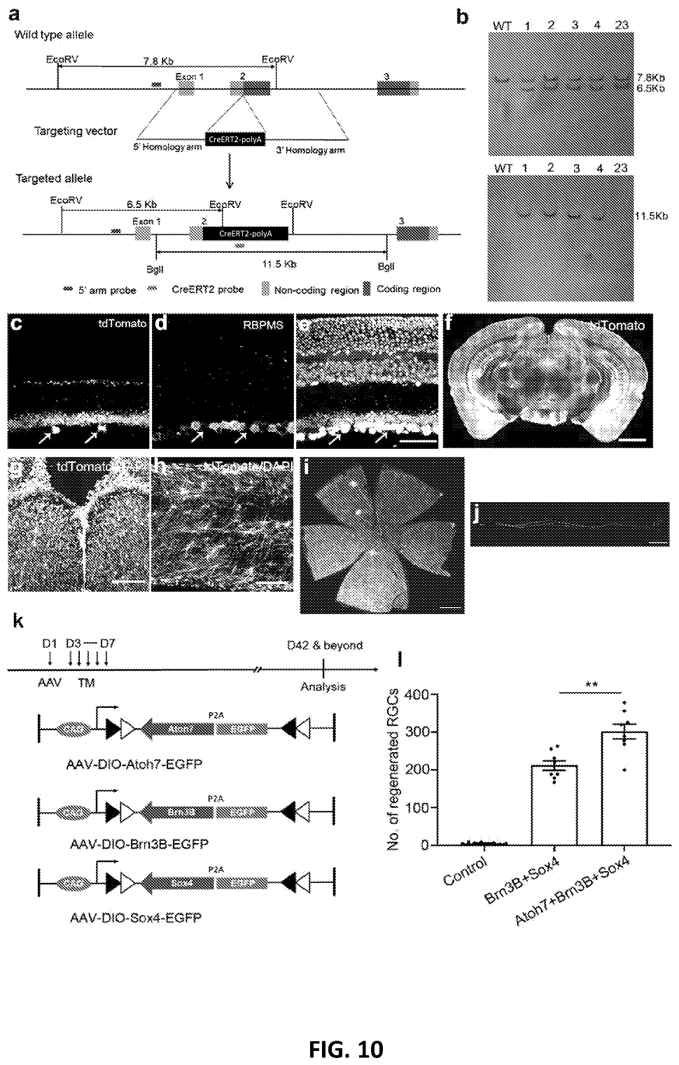

Reprogram Prokr2⁺ displaced amacrine interneurons into RGCs. We contemplated whether other retinal neurons could be reprogrammed into RGCs too. Displaced amacrine interneurons could serve as a better cellular source for RGC replacement, since they are located in the RGC layer. To test if this neuronal subtype could be reprogrammed into RGCs, we generated a Prokr2^{CreERT2} knock-in mouse line (FIG. 10a, b). The Prokr2^{CreERT2} mice express the tamoxifen-inducible CreERT2 recombinase under the endogenous transcriptional control of the Prokr2 gene, which is expressed in a subgroup of displaced amacrine interneurons. As expected, in adult Prokr2^{CreERT2}; Rosa26-tdTomato mice treated with tamoxifen, tdTomato⁺ cells are located in the retinal ganglion cell layer. They do not have optic projections and do not express the RGC maker RBPMS (FIG. 4a, b and FIG. 10c-e).

In addition to being expressed in the retina, Prokr2 is also expressed in cells of the optic nerve and the brain (FIG. 10f-h). This prevented us from using the Rosa26-tdTomato reporter to track axons of regenerated RGCs. To overcome this obstacle, we labeled regenerated RGCs by co-expressing EGFP with transcription factors during programming (FIG. 10k). We used two combinations of transcription factors (Brn3B+Sox4 and Atoh7+Brn3B+Sox4) for reprogramming, and found that both combinations could efficiently reprogram Prokr2⁺ displaced amacrine interneurons into RGCs (FIG. 4c, d). However, unlike in Lgr5⁺ amacrine interneurons, inclusion of Atoh7 to the Brn3B+Sox4 combination dramatically enhanced reprogramming efficiency (FIG. 10l). Prokr2⁺ displaced amacrine interneuron-derived RGCs also extended axonal projections into the optic nerve and various brain visual targets (FIG. 4e-k). Thus, these results demonstrated that RGCs could be regenerated by reprogramming multiple retinal neuron subtypes in vivo.

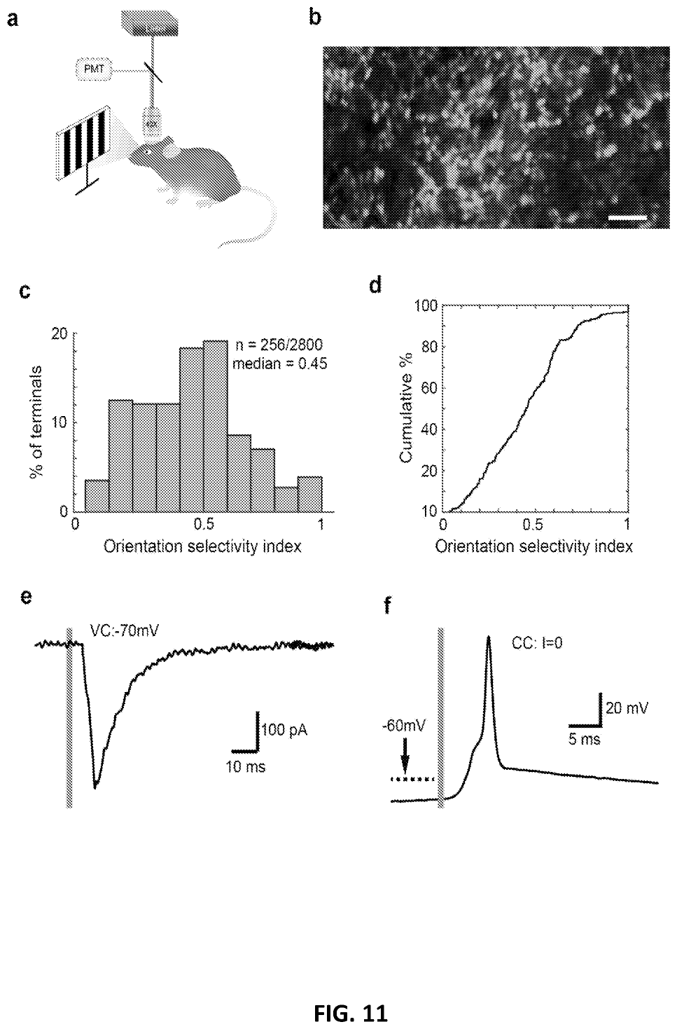

Regenerated RGCs convey visual information to the brain. To investigate whether regenerated RGCs could respond to visual stimulation and convey visual information to downstream targets in the brain, we labeled regenerated RGCs with the calcium indicator GCamp6f in Lgr5^{EGFP-IRES-CreERT2} mice by adding AAV-DIO-GCamp6f to the reprogramming cocktail. We then exposed SC of anesthetized mice six weeks after viral injection, and used in vivo functional calcium imaging to measure the visually evoked calcium dynamics of regenerated RGC axon terminals (FIG. 11a).

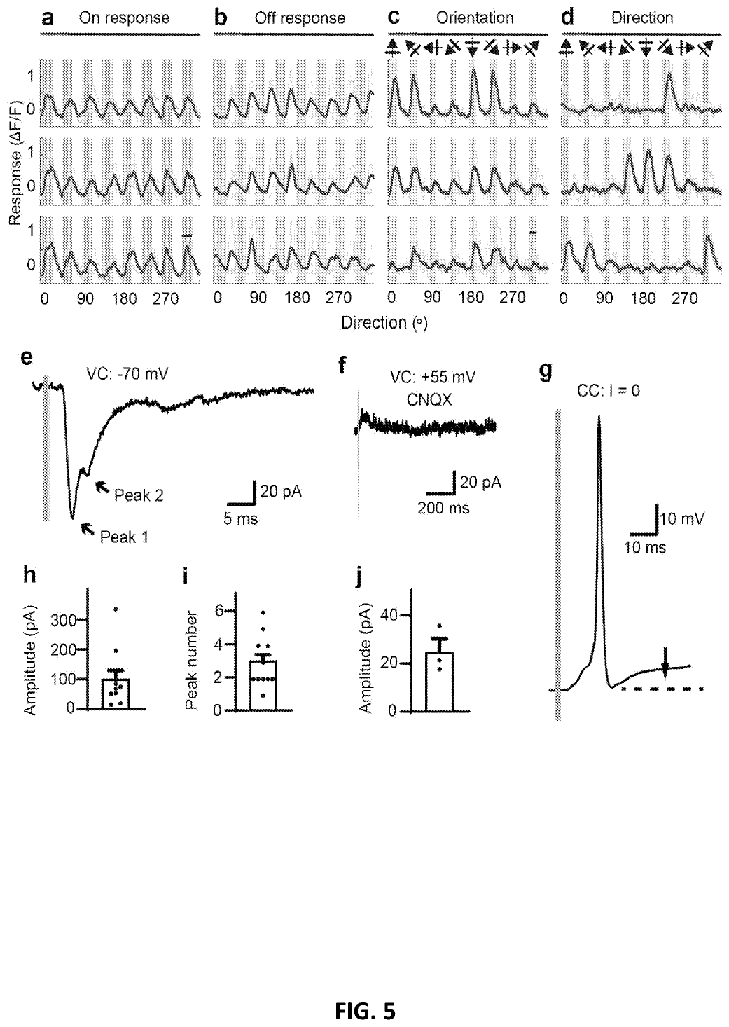

When mice were presented with drifting gratings, individual RGC boutons along the axonal arborization in SC exhibited stimulus-evoked calcium signal (FIG. 11b), indicating that regenerated RGCs could respond to visual stimulation and transmit visual signals to the brain. Visually responsive boutons could be classified into distinct categories based on their response patterns. Boutons responded differently to on and off of stimulation, as well as to the orientation and direction of drifting gratings (FIG. 5a-d and 11c, d). Together, these data suggest that regenerated RGCs are capable of conveying visual information to the brain, and functionally distinct RGC subtypes could be generated by in vivo reprogramming.

Regenerated RGCs establish functional synaptic connections with postsynaptic neurons. To investigate whether regenerated RGCs could transmit neuronal signals to postsynaptic neurons in the brain, we expressed Channelrhodopsin-2 (ChR2) in regenerated RGCs in Lgr5^{EGFP-IBES-CreERT2}; Rosa26-tdTomato mice, and used whole-cell patch recording to detect light-evoked postsynaptic responses of SC neurons on brain slices 8-10 weeks after viral injection.

When axon terminals of regenerated RGCs were stimulated with light, AMPA receptor-mediated excitatory postsynaptic currents (EPSCs) were detected in SC neurons. A single light impulse evoked AMPA receptor-mediated EPSCs with multiple peaks (FIG. 5e, h and i), suggesting that regenerated RGC axons formed multi-input synapses with SC neurons and activated AMPA glutamatergic receptors. NMDA receptor-mediated EPSCs and action potential were also detected in postsynaptic SC neurons after light stimulation (FIG. 5f, g and j). Furthermore, the response of SC neurons to regenerated ChR2-expressing RGCs is comparable to that of normal RGCs expressing ChR2 (FIG. 11e, f). Together, these results suggest that, in response to light stimulation, regenerated RGC axon terminals release glutamate as neurotransmitter and establish functional synaptic connections with SC neurons.

Regenerate functional RGCs in a mouse model of glaucoma. We next asked whether regenerated RGCs could repair visual circuits under diseased conditions. We are particularly interested in determining whether regenerated RGCs could still send axons to appropriate brain targets, and rebuild the retina-brain connection, when original RGCs and their axons have undergone degeneration.

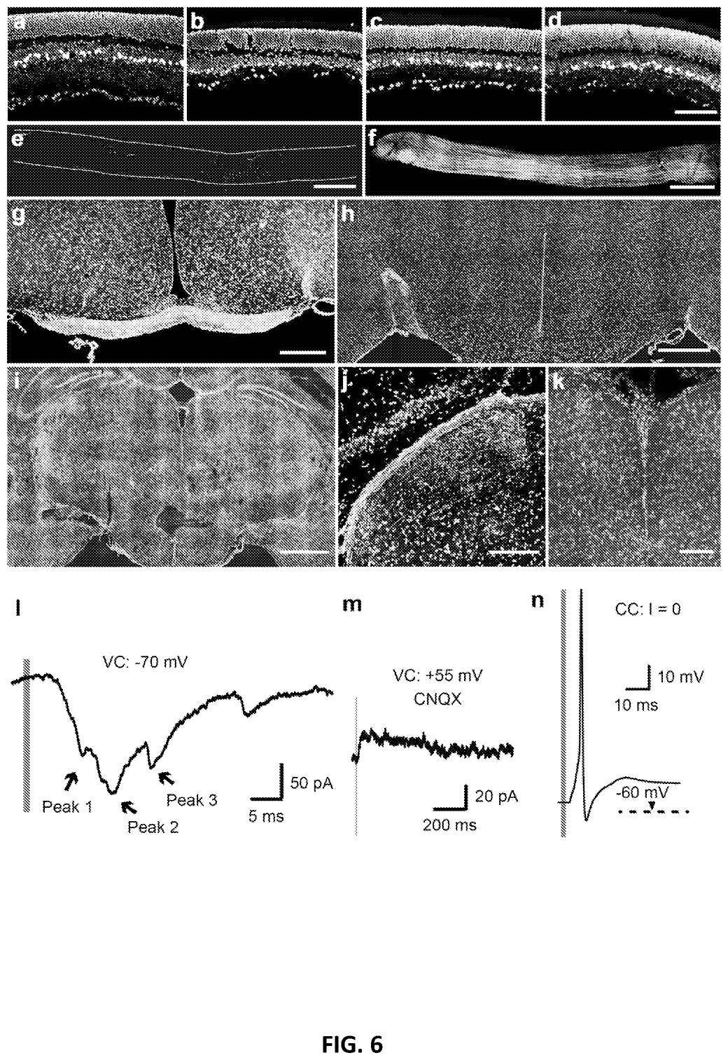

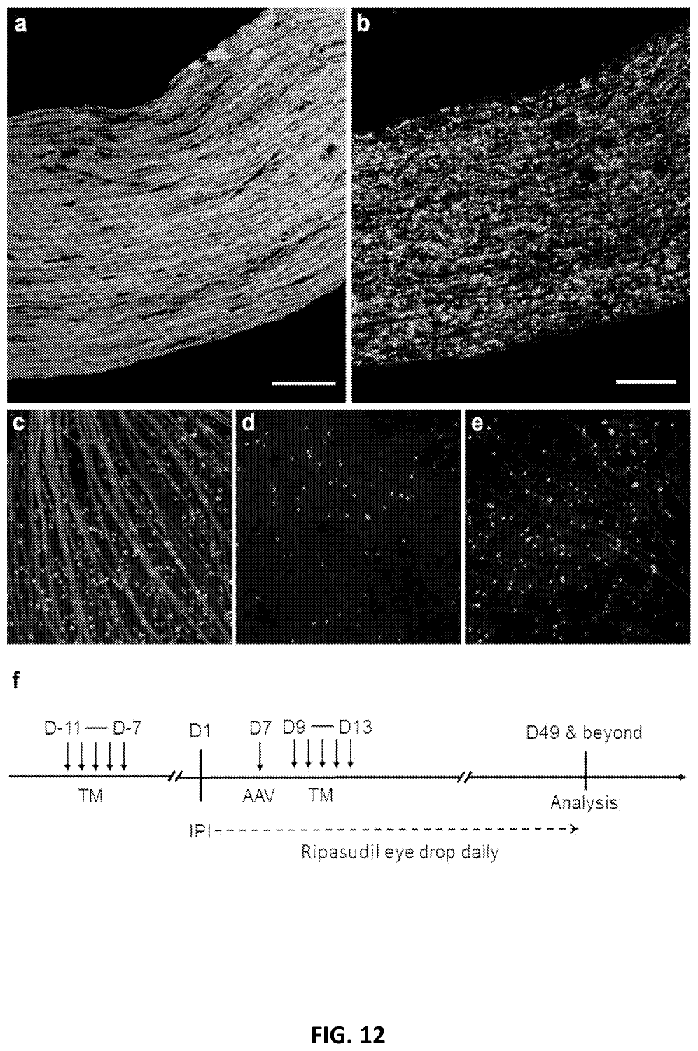

We used an intraocular pressure increase-induced glaucoma model to damage RGCs and their axons, and optimized a condition that could cause degeneration of all RGC axons within the optic nerve and significant loss of RGC cell bodies in the retina (FIG. 12a-e). However, this damage condition also caused dramatic loss of Lgr5⁺ amacrine cells seven days after intraocular pressure increase (FIG. 6b). Therefore, we searched for reagents that could protect Lgr5⁺ amacrine cells, and found that the Rock inhibitor Ripasudil could efficiently preserve Lgr5⁺ amacrine cells (FIG. 6c).

We devised a protocol that combined neuronal protection with in vivo reprogramming, to test if newly generated RGCs could repair damaged visual circuitry in the Lgr5^{EGFP-IRES-CreERT2}; Rosa26-tdTomato mice (FIG. 12f). We damaged both eyes of Lgr5^{EGFP-IRES-CreERT2}; Rosa26-tdTomato mice that have received tamoxifen feeding to label Lgr5⁺ amacrine cells with the tdTomato reporter. After damage, both eyes were treated with Ripasudil once a day, and seven days later, the left eye was injected with AAV-DIO-EGFP as control, whereas the right eye was injected with a combination of AAVs expressing transcription factors including Brn3B, Sox4 and Atoh7, to reprogram Lgr5⁺ amacrine interneurons. Six weeks after viral injection, mice were sacrificed for analysis. In the left eye injected with AAV-DIO-EGFP, no RGCs were regenerated, since no tdTomato⁺ RGC axons could be detected in the left optic nerve (FIG. 6e). In contrast, overexpression of Brn3B, Sox4 and Atoh7 reprogrammed Lgr5⁺ amacrine cells into RGCs, and regenerated RGCs projected tdTomato⁺ axons into the optic nerve and various brain visual targets of the contralateral side (FIG. 6f-k).

Regenerated RGCs established functional synaptic connections with postsynaptic brain neurons under diseased conditions. Light-evoked postsynaptic responses were detected in SC neurons on brain slices, where all RGC axon terminals were from regenerated RGCs after original ones had been damaged (FIG. 6l-n). Together, these results demonstrate that regenerated RGCs could reconnect the retina to the brain and transmit visual information to postsynaptic neurons even under diseased conditions.

These results demonstrate that functional RGCs can be generated in adult mammals by in vivo reprogramming of fully differentiated retinal interneurons. By ectopic expression of essential transcription factors, both amacrine and displaced amacrine interneurons can be precisely reprogrammed into RGCs, and newly generated RGCs integrate into the visual circuitry and transmit visual information to the brain. Although in vivo neuronal identity reprogramming has been achieved in other regions of the central nervous system (CNS), successful conversion between neuronal subtypes was only restricted to the first postnatal week, or with limited success in adult mice when the chemical compound valproic acid is present. In contrast, this example demonstrated that, even without chemical-stimulant, retinal neuronal identity switching can be achieved in adulthood, and successful reprogramming even triggers migration of amacrine interneurons from the inner nuclear layer to the RGC layer. These results show that neurons exhibit surprisingly unexpected identity plasticity, which could be harnessed for regenerative purposes.

The combination of Brn3B and Sox4 efficiently reprogramed both Lgr5⁺ amacrine interneurons and Prokr2⁺ displaced amacrine interneurons into RGCs, indicating that these two transcription factors are sufficient for RGC fate determination. Including Atoh7 to the Brn3B+Sox4 combination significantly improved the efficiency of regenerating RGCs from Prokr2⁺ displaced amacrine cells, although it did not improve Lgr5⁺ amacrine cell reprogramming This suggests that direct cell lineage reprogramming is affected by intrinsic properties of source cells.

Regenerated RGCs connect retina to brain by long-distance projection of axons into various brain visual areas, even in animals where the original RGCs and axons have been damaged. These findings reveal that the adult mammalian visual system remains a remarkable ability of reconnecting neural circuits.

### Example 2

### Rejuvenation of Degenerated RGCs

This example shows that degenerated RGCs can also be reactivated by the transcription factors to grow functional axons again.

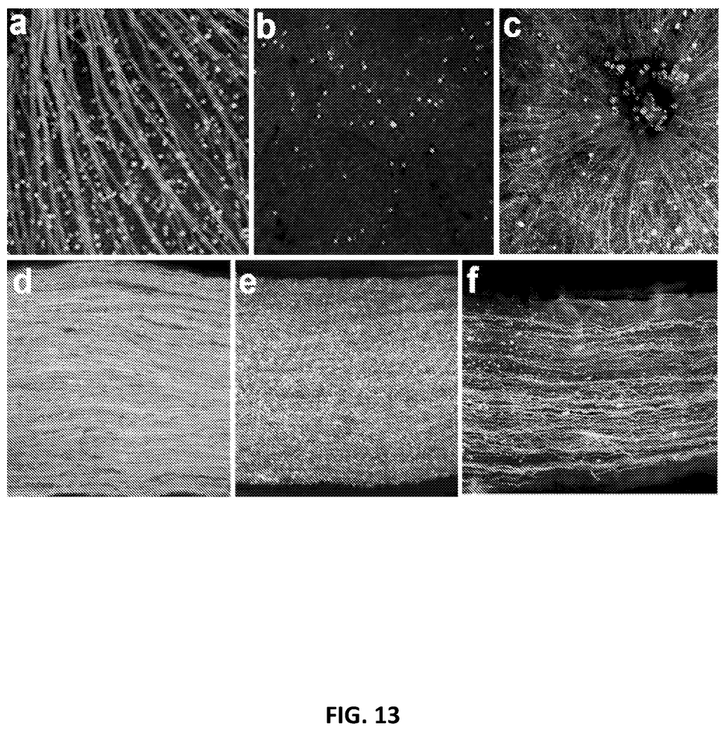

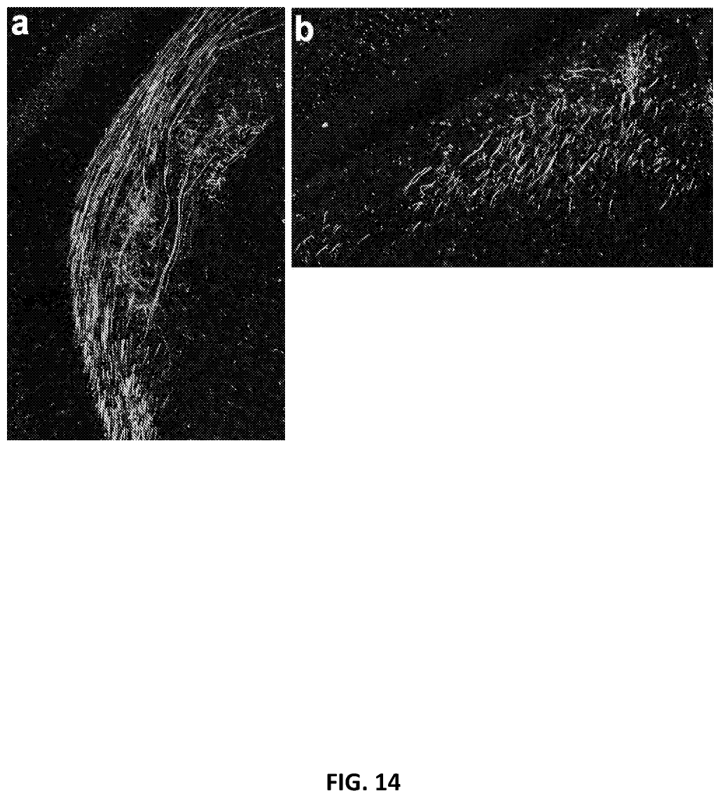

In this example, the increase of intraocular pressure was used to trigger apoptosis of retinal ganglion cells (RGCs), leading to degeneration of their axons, in PV-CreERT2; Rosa26-tdTomato mice. In this animal model, expression of three transcription factors (Atoh7+Brn3B+Sox4) in survived RGCs stimulated these cells to regrow (regenerate) axons (FIG. 13). Also important, the regenerated RGC axons projected into the optic nerve and reached visual areas with the brain (FIG. 14).

The results, therefore, demonstrate that combinations of transcription factors not only can reprogram interneuron cells into regenerated RGCs, they can also rejuvenate degenerated, damaged, injured, or aged RGCs. Accordingly, when these transcription factors are administered to a subject that desires visual repair, restoration, or improvement, they can work in concert on both the interneurons and the RGCs to achieve the desired therapeutic effect.

Although this disclosure has been described with reference to the disclosed embodiments, those skilled in the art will readily appreciate that the specific examples and studies detailed above are only illustrative of this disclosure. It should be understood that various modifications can be made without departing from the spirit of this disclosure. Accordingly, this disclosure is limited only by the following claims.

What is claimed is:

1. A method for preparing a mammalian cell responsive to visual signals, comprising increasing the biological activity, in a retinal neuron cell, of one or more genes selected from the group consisting of:
POU class 4 homeobox 2 (Brn3B)
SRY-box transcription factor 4 (Sox4)
Atonal BHLH Transcription Factor 7 (Atoh7),
SRY-Box Transcription Factor 11 (Sox11), and
ISL LIM homeobox 1 (Ils1).

2. The method of claim 1, wherein the one or more genes comprise Brn3B and Sox4.

3. The method of claim 2, wherein the one or more genes further comprise Atoh7.

4. The method of claim 1, wherein the retinal neuron cell is a retinal interneuron cell selected from the group consisting of an amacrine cell, a horizontal cell, and a bipolar cell, or is a degenerated, damaged, or aged retinal ganglion cell (RGC).

5. The method of claim 1, wherein the retinal neuron cell is a Lgr5⁺ amacrine cell.

6. The method of claim 1, wherein the retinal neuron cell is a Prokr2⁺ displaced amacrine cell.

7. A method for improving the function of a retinal ganglion cell (RGC), comprising increasing the biological activity, in the RGC, of one or more genes selected from the group consisting of Atoh7, Brn3B, Sox4, Sox11, and Ils1.

8. The method of claim 7, wherein the RGC is a degenerated, damaged, aged, or normal retinal ganglion cell (RGC).

9. The method of claim 1, wherein increasing the biological activity of the one or more genes comprises introducing to the retinal neuron cell one or more polynucleotide encoding the genes.

10. The method of claim 9, wherein the one or more polynucleotide is cDNA.

11. The method of claim 10, wherein the one or more polynucleotide is provided in a plasmid or viral vector.

12. The method of claim 1, wherein the retinal neuron cell is in vivo in a subject having visual impairment.

13. A method for treating visual impairment or blindness in a subject in need thereof, comprising administering to the retina of the subject an agent capable of increasing the biological activity of one or more genes selected from the group consisting of Brn3B, Sox4, Atoh7, Sox11, and Ils1.

14. The method of claim 13, wherein the one or more genes comprise Brn3B and Sox4.

15. The method of claim 13, wherein the visual impairment or blindness is caused by degenerated retinal ganglion cells (RGCs).

16. The method of claim 13, wherein the visual impairment or blindness is associated with a condition selected from the group consisting of optic neuropathy, including glaucoma, hereditary optic neuropathy, and disorders caused by toxins, nutritional defects and trauma.


SEQUENCE LISTING (27 sequences)

SEQ ID NO: 1 - protein, 409 aa, Homo sapiens
MMMMSLNSKQAFSMPHGGSLHVEPKYSALHSTSPGSSAPIAPSASSPSSSSNAGGGGGGGGGGGGGGGRSSSSSSSGSSGGGGSEAMRRACLPTPPSNIFGGLDESLLARAEALAAVDIVSQSKSHHHHPPHHSPFKPDATYHTMNTIPCTSAASSSSVPISHPSALAGTHHHHHHHHHHHHQPHQALEGELLEHLSPGLALGAMAGPDGAVVSTPAHAPHMATMNPMHQAALSMAHAHGLPSHMGCMSDVDADPRDLEAFAERFKQRRIKLGVTQADVGSALANLKIPGVGSLSQSTICRFESLTLSHNNMIALKPILQAWLEEAEKSHREKLTKPELFNGAEKKRKRTSIAAPEKRSLEAYFAIQPRPSSEKIAAIAEKLDLKKNVVRVWFCNQRQKQKRMKYSAGI

SEQ ID NO: 2 - DNA, 1230 bp, Homo sapiens
  source 1..1230: mol_type = other DNA
atgatgatgatgtccctgaacagcaagcaggcgtttagcatgccgcacggcggcagcctgcacgtggagcccaagtactcggcactgcacagcacctcgccgggctcctcggctcccatcgcgccctcggccagctcccccagcagctcgagcaacgctggtggtggcggcggcggcggcggcggcggcggcggcggcggaggccgaagcagcagctccagcagcagtggcagcagcggcggcgggggctcggaggctatgcggagagcctgtcttccaaccccaccgagcaatatattcggcgggctggatgagagtctgctggcccgcgccgaggctctggcagccgtggacatcgtctcccagagcaagagccaccaccaccatccaccccaccacagccccttcaaaccggacgccacctaccacactatgaataccatcccgtgcacgtcggccgcctcttcttcatcggtgcccatctcgcacccttccgcgttggcgggcacgcaccaccaccaccaccatcaccaccaccaccaccaccaaccgcaccaggcgctggagggcgagctgctggagcacctgagtcccgggctggccctgggcgctatggcgggccccgacggcgctgtggtgtccacgccggctcacgcgccgcacatggccaccatgaaccccatgcaccaagcagcgctcagcatggcccacgcgcacgggctgccgtcgcacatgggctgcatgagcgacgtggacgccgacccgcgggacctggaggcattcgccgagcgcttcaagcagcgacgcatcaagctgggggtgacccaggcagatgtgggctccgcgctggccaacctcaagatccccggcgtgggctcgcttagccagagcaccatctgcaggttcgagtccctcacactgtcccacaataatatgatcgcgctcaaacccatcctgcaggcatggctcgaggaggccgagaagtcccaccgcgagaagctcaccaagcctgaactcttcaatggcgcggagaagaagcgcaagcgcacgtccatcgctgcgccagagaagcgctcgctcgaagcctactttgccattcagcctcggccctcctctgaaaagatcgccgccatcgcggagaagctggacctgaagaaaaacgtggtgcgcgtctggttctgcaaccagaggcagaaacagaaaagaatgaaatattccgccggcatttag

SEQ ID NO: 3 - protein, 411 aa, Mus musculus
MMMMSLNSKQAFSMPHAGSLHVEPKYSALHSASPGSSAPAAPSASSPSSSSNAGGGGGGGGGGGGGGRSSSSSSSGSGGSGGGGGSEAMRRACLPTPPSNIFGGLDESLLARAEALAAVDIVSQSKSHHHHPPHHSPFKPDATYHTMNTIPCTSAASSSSVPISHPSALAGTHHHHHHHHHHHHQPHQALEGELLEHLSPGLALGAMAGPDGTVVSTPAHAPHMATMNPMHQAALSMAHAHGLPSHMGCMSDVDADPRDLEAFAERFKQRRIKLGVTQADVGSALANLKIPGVGSLSQSTICRFESLTLSHNNMIALKPILQAWLEEAEKSHREKLTKPELFNGAEKKRKRTSIAAPEKRSLEAYFAIQPRPSSEKIAAIAEKLDLKKNVVRVWFCNQRQKQKRMKYSAGI

SEQ ID NO: 4 - DNA, 1236 bp, Mus musculus
  source 1..1236: mol_type = other DNA
atgatgatgatgtccctgaacagcaagcaggcgttcagcatgcctcacgcaggcagcctgcacgtggagcccaagtactcggcgctacacagtgcctccccgggctcctctgcgcccgcggcgccctcggccagttcccctagcagctccagcaacgctggcggcggcggcggtggcggcggaggcggaggcggcggcggccggagcagcagttccagcagcagtggcagcggcggcagcggcggcggcgggggctcggaggcgatgcggagagcttgtcttccaaccccaccgagcaatatattcggcgggctggatgagagtctgctggcccgtgccgaggctctggccgccgtggacatcgtctcccagagtaagagccaccaccaccatccgccccaccacagccccttcaagccggacgccacttaccacaccatgaacaccatcccgtgcacgtcggcagcctcctcttcttctgtgcccatctcgcacccgtccgctctggctggcacccatcaccaccaccaccaccaccatcaccaccatcaccagccgcaccaggcgctggagggcgagctgcttgagcacctaagccccgggctggccctgggagctatggcgggccccgacggcacggtggtgtccactccggctcacgcaccacacatggccaccatgaaccccatgcaccaagcagccctgagcatggcccacgcacatgggctgccctcgcacatgggctgcatgagcgacgtggatgcagacccgcgggacctggaggcgttcgccgagcgtttcaagcagcgacgcatcaagctgggagtgacccaggcagatgtgggctcggcgctggccaacctcaagatcccgggcgtgggctcgctcagccagagcaccatctgcaggtttgagtctctcacgctgtcacacaacaacatgatcgcgctcaagcccatcctgcaggcgtggctggaggaagctgagaaatcccaccgcgagaagctcactaagccggagctcttcaatggcgcggagaagaagcgcaagcgcacgtccatcgcggcgccggagaagcgctctctggaagcctacttcgccatccagccaaggccctcctcggagaagatcgcggccatcgccgaaaagctggatctcaagaaaaatgtggtgcgcgtctggttctgcaaccagaggcagaaacagaagagaatgaaatactctgccggcatttag

SEQ ID NO: 5 - protein, 474 aa, Homo sapiens
MVQQTNNAENTEALLAGESSDSGAGLELGIASSPTPGSTASTGGKADDPSWCKTPSGHIKRPMNAFMVWSQIERRKIMEQSPDMHNAEISKRLGKRWKLLKDSDKIPFIREAERLRLKHMADYPDYKYRPRKKVKSGNANSSSSAAASSKPGEKGDKVGGSGGGGHGGGGGGGSSNAGGGGGGASGGGANSKPAQKKSCGSKVAGGAGGGVSKPHAKLILAGGGGGGKAAAAAAASFAAEQAGAAALLPLGAAADHHSLYKARTPSASASASSAASASAALAAPGKHLAEKKVKRVYLFGGLGTSSSPVGGVGAGADPSDPLGLYEEEGAGCSPDAPSLSGRSSAASSPAAGRSPADHRGYASLRAASPAPSSAPSHASSSASSHSSSSSSSGSSSSDDEFEDDLLDLNPSSNFESMSLGSFSSSSALDRDLDFNFEPGSGSHFEFPDYCTPEVSEMISGDWLESSISNLVFTY

SEQ ID NO: 6 - DNA, 1425 bp, Homo sapiens
  source 1..1425: mol_type = other DNA
atggtgcagcaaaccaacaatgccgagaacacggaagcgctgctggccggcgagagctcggactcgggcgccggcctcgagctgggaatcgcctcctcccccacgcccggctccaccgcctccacgggcggcaaggccgacgacccgagctggtgcaagaccccgagtgggcacatcaagcgacccatgaacgccttcatggtgtggtcgcagatcgagcggcgcaagatcatggagcagtcgcccgacatgcacaacgccgagatctccaagcggctgggcaaacgctggaagctgctcaaagacagcgacaagatccctttcattcgagaggcggagcggctgcgcctcaagcacatggctgactaccccgactacaagtaccggcccaggaagaaggtgaagtccggcaacgccaactccagctcctcggccgccgcctcctccaagccgggggagaagggagacaaggtcggtggcagtggcgggggcggccatgggggcggcggcggcggcgggagcagcaacgcggggggaggaggcggcggtgcgagtggcggcggcgccaactccaaaccggcgcagaaaaagagctgcggctccaaagtggcgggcggcgcgggcggtggggttagcaaaccgcacgccaagctcatcctggcaggcggcggcggcggcgggaaagcagcggctgccgccgccgcctccttcgccgccgaacaggcgggggccgccgccctgctgcccctgggcgccgccgccgaccaccactcgctgtacaaggcgcggactcccagcgcctcggcctccgcctcctcggcagcctcggcctccgcagcgctcgcggccccgggcaagcacctggcggagaagaaggtgaagcgcgtctacctgttcggcggcctgggcacgtcgtcgtcgcccgtgggcggcgtgggcgcgggagccgaccccagcgaccccctgggcctgtacgaggaggagggcgcgggctgctcgcccgacgcgcccagcctgagcggccgcagcagcgccgcctcgtcccccgccgccggccgctcgcccgccgaccaccgcggctacgccagcctgcgcgccgcctcgcccgccccgtccagcgcgccctcgcacgcgtcctcctcggcctcgtcccactcctcctcttcctcctcctcgggctcctcgtcctccgacgacgagttcgaagacgacctgctcgacctgaaccccagctcaaactttgagagcatgtccctgggcagcttcagttcgtcgtcggcgctcgaccgggacctggattttaacttcgagcccggctccggctcgcacttcgagttcccggactactgcacgcccgaggtgagcgagatgatctcgggagactggctcgagtccagcatctccaacctggttttcacctactga

SEQ ID NO: 7 - protein, 440 aa, Mus musculus
MVQQTNNAENTEALLAGESSDSGAGLELGIASSPTPGSTASTGGKADDPSWCKTPSGHIKRPMNAFMVWSQIERRKIMEQSPDMHNAEISKRLGKRWKLLKDSDKIPFIQEAERLRLKHMADYPDYKYRPRKKVKSGNAGAGSAATAKPGEKGDKVAGSSGHAGSSHAGGGAGGSSKPAPKKSCGPKVAGSSVGKPHAKLVPAGGSKAAASFSPEQAALLPLGEPTAVYKVRTPSAATPAASSSPSSALATPAKHPADKKVKRVYLFGSLGASASPVGGLGASADPSDPLGLYEDGGPGCSPDGRSLSGRSSAASSPAASRSPADHRGYASLRAASPAPSSAPSHASSSLSSSSSSSSGSSSSDDEFEDDLLDLNPSSNFESMSLGSFSSSSALDRDLDFNFEPGSGSHFEFPDYCTPEVSEMISGDWLESSISNLVFTY

SEQ ID NO: 8 - DNA, 1323 bp, Mus musculus
  source 1..1323: mol_type = other DNA
atggtacaacagaccaacaacgcggagaacactgaggctctgctggccggggagagctcggactcgggcgccggcctggagctgggcatcgcgtcctccccgacgcctggctccaccgcgtcgacgggcggcaaggcggacgaccccagctggtgcaagacgcccagtggccacatcaagcggcccatgaacgcctttatggtgtggtcgcagatcgagcggcgcaagatcatggagcagtcgcccgacatgcacaacgccgagatctccaagcggctaggcaaacgctggaagctgctcaaggacagcgacaagattccgttcatccaggaggcggagcggctgcgcctcaagcacatggctgactaccctgactacaagtaccggccgcgaaagaaggtgaagtcgggcaacgcgggcgcgggatcggcggccacagccaagccaggggagaagggcgacaaggtcgcgggcagcagcggccacgcgggaagcagccacgcggggggtggcgcgggcggcagctccaagcccgcgcccaagaagagctgtggccccaaggtggcgggcagctcggtcggcaagccccacgctaagctggtcccggcgggcggcagcaaggcggctgcatcgttctctccagagcaagctgccctgctgcccctgggggagcccacggccgtctacaaggtgcggactcccagtgcggccactccggccgcctcctcctcgccgtccagtgcgctggccaccccagccaaacaccctgccgacaagaaagtgaagcgcgtctacctgtttggaagcctgggcgcttcggcgtctcccgtcgggggcctgggagcgagcgccgaccccagtgatccactggggttgtacgaagatggaggcccgggatgctcgcccgatggccggagtctgagcggccgcagcagcgcagcatcatcgccagccgccagccgatcgcccgctgaccaccgcggctacgccagcctacgcgcagcctcgcccgccccgtccagcgcgccctcgcacgcgtcctcctcgctctcctcgtcctcttcctcctcctcgggctcttcgtcgtccgacgacgagttcgaagacgacctgctcgacctgaaccccagctcaaactttgagagcatgtccctgggcagtttcagctcctcatcggcgctcgatcgggacctggattttaacttcgaacccggctcaggctcccacttcgaattcccggactattgcacgcccgaggtgagcgagatgatctcgggagattggctggagtccagcatctctaacctggtcttcacctactga

SEQ ID NO: 9 - protein, 152 aa, Homo sapiens
MKSCKPSGPPAGARVAPPCAGGTECAGTCAGAGRLESAARRRLAANARERRRMQGLNTAFDRLRRVVPQWGQDKKLSKYETLQMALSYIMALTRILAEAERFGSERDWVGLHCEHFGRDHYLPFPGAKLPGESELYSQRLFGFQPEPFQMAT

SEQ ID NO: 10 - DNA, 459 bp, Homo sapiens
  source 1..459: mol_type = other DNA
atgaagtcctgcaagcccagcggcccgccggcgggagcgcgcgttgcacccccgtgcgcgggcggcaccgagtgcgcgggcacgtgcgccggggccgggcggctggagagcgcggcgcgcaggcgcctggcggccaacgcgcgcgagcgccgccgcatgcaggggctcaacactgccttcgaccgcttacgcagggtggttccccagtggggccaggataaaaagctgtccaagtacgagaccctgcagatggccctgagctacatcatggctctgacccggatcctggccgaggccgagcgattcggctcggagcgggactgggtgggtctccactgtgagcacttcggccgcgaccactacctcccgttcccgggcgcgaagctgccgggcgagagcgagctgtacagccagagactcttcggcttccagcccgagcccttccagatggccacctag

SEQ ID NO: 11 - protein, 149 aa, Mus musculus
MKSACKPHGPPAGARGAPPCAGAAERAVSCAGPGRLESAARRRLAANARERRRMQGLNTAFDRLRRVVPQWGQDKKLSKYETLQMALSYIIALTRILAEAERDWVGLRCEQRGRDHPYLPFPGARLQVDPEPYGQRLFGFQPEPFPMAS

SEQ ID NO: 12 - DNA, 450 bp, Mus musculus
  source 1..450: mol_type = other DNA
atgaagtcggcctgcaaaccccacggccctccggcgggagctcgcggcgcgcccccgtgcgcgggcgcagccgagcgcgcggtctcgtgcgcggggcccgggcggctggagagcgcggcgcgcaggcgtctggcggccaacgcgcgcgagcggcgccgcatgcaggggctgaacacggcgttcgaccggctgcgcagggtggtgccgcagtggggccaggacaagaagctgtccaagtacgagacactgcagatggcgctcagctacatcatcgcgctcacccgcatcctagccgaagccgagcgggactgggtcgggctgcgctgcgagcagcggggccgcgatcacccctacctccctttcccgggtgctaggctccaggtagaccctgagccctatgggcagaggctcttcggcttccagccggagcccttccccatggccagctaa

SEQ ID NO: 13 - protein, 129 aa, Mus musculus
MKSACKPHGPPAGARGAPPCAGAAERAVSCAGPGRLESAARRRLAANARERRRMQGLNTAFDRLRRVVPQWGQDKKLSKYETLQMALSYIIALTRILAEAERDWVGLRCEQRGRDHPYLPFPGARLQVS

SEQ ID NO: 14 - DNA, 390 bp, Mus musculus
  source 1..390: mol_type = other DNA
atgaagtcggcctgcaaaccccacggccctccggcgggagctcgcggcgcgcccccgtgcgcgggcgcagccgagcgcgcggtctcgtgcgcggggcccgggcggctggagagcgcggcgcgcaggcgtctggcggccaacgcgcgcgagcggcgccgcatgcaggggctgaacacggcgttcgaccggctgcgcagggtggtgccgcagtggggccaggacaagaagctgtccaagtacgagacactgcagatggcgctcagctacatcatcgcgctcacccgcatcctagccgaagccgagcgggactgggtcgggctgcgctgcgagcagcggggccgcgatcacccctacctccctttcccgggtgctaggctccaggtttcatga

SEQ ID NO: 15 - protein, 441 aa, Homo sapiens
MVQQAESLEAESNLPREALDTEEGEFMACSPVALDESDPDWCKTASGHIKRPMNAFMVWSKIERRKIMEQSPDMHNAEISKRLGKRWKMLKDSEKIPFIREAERLRLKHMADYPDYKYRPRKKPKMDPSAKPSASQSPEKSAAGGGGGSAGGGAGGAKTSKGSSKKCGKLKAPAAAGAKAGAGKAAQSGDYGGAGDDYVLGSLRVSGSGGGGAGKTVKCVFLDEDDDDDDDDDELQLQIKQEPDEEDEEPPHQQLLQPPGQQPSQLLRRYNVAKVPASPTLSSSAESPEGASLYDEVRAGATSGAGGGSRLYYSFKNITKQHPPPLAQPALSPASSRSVSTSSSSSSGSSSGSSGEDADDLMFDLSLNFSQSAHSASEQQLGGGAAAGNLSLSLVDKDLDSFSEGSLGSHFEFPDYCTPELSEMIAGDWLEANFSDLVFTY

SEQ ID NO: 16 - DNA, 1326 bp, Homo sapiens
  source 1..1326: mol_type = other DNA
atggtgcagcaggcggagagcttggaagcggagagcaacctgccccgggaggcgctggacacggaggagggcgaattcatggcttgcagcccggtggccctggacgagagcgacccagactggtgcaagacggcgtcgggccacatcaagcggccgatgaacgcgttcatggtatggtccaagatcgaacgcaggaagatcatggagcagtctccggacatgcacaacgccgagatctccaagaggctgggcaagcgctggaaaatgctgaaggacagcgagaagatcccgttcatccgggaggcggagcggctgcggctcaagcacatggccgactaccccgactacaagtaccggccccggaaaaagcccaaaatggacccctcggccaagcccagcgccagccagagcccagagaagagcgcggccggcggcggcggcgggagcgcgggcggaggcgcgggcggtgccaagacctccaagggctccagcaagaaatgcggcaagctcaaggcccccgcggccgcgggcgccaaggcgggcgcgggcaaggcggcccagtccggggactacgggggcgcgggcgacgactacgtgctgggcagcctgcgcgtgagcggctcgggcggcggcggcgcgggcaagacggtcaagtgcgtgtttctggatgaggacgacgacgacgacgacgacgacgacgagctgcagctgcagatcaaacaggagccggacgaggaggacgaggaaccaccgcaccagcagctcctgcagccgccggggcagcagccgtcgcagctgctgagacgctacaacgtcgccaaagtgcccgccagccctacgctgagcagctcggcggagtcccccgagggagcgagcctctacgacgaggtgcgggccggcgcgacctcgggcgccgggggcggcagccgcctctactacagcttcaagaacatcaccaagcagcacccgccgccgctcgcgcagcccgcgctgtcgcccgcgtcctcgcgctcggtgtccacctcctcgtccagcagcagcggcagcagcagcggcagcagcggcgaggacgccgacgacctgatgttcgacctgagcttgaatttctctcaaagcgcgcacagcgccagcgagcagcagctggggggcggcgcggcggccgggaacctgtccctgtcgctggtggataaggatttggattcgttcagcgagggcagcctgggctcccacttcgagttccccgactactgcacgccggagctgagcgagatgatcgcgggggactggctggaggcgaacttctccgacctggtgttcacatattga

SEQ ID NO: 17 - protein, 395 aa, Mus musculus
MVQQAESSEAESNLPRDALDTEEGEFMACSPVALDESDPDWCKTASGHIKRPMNAFMVWSKIERRKIMEQSPDMHNAEISKRLGKRWKMLKDSEKIPFIREAERLRLKHMADYPDYKYRPRKKPKTDPAAKPSAGQSPDKSAAGAKAAKGPGKKCAKLKAPAGKAGAGKAAQPGDCAAGKAAKCVFLDDDDEDDDEDDELQLRPKPDADDDDDEPAHSHLLPPPTQQQPPQLLRRYSVAKVPASPTLSSAAESPEGASLYDEVRAGGRLYYSFKNITKQQPPPAPPALSPASSRCVSTSSSSGSSSGSGAEDADDLMFDLSLNFSQGAHSACEQPLGAGAAGNLSLSLVDKDLDSFSEGSLGSHFEFPDYCTPELSEMIAGDWLEANFSDLVFTY

SEQ ID NO: 18 - DNA, 1188 bp, Mus musculus
  source 1..1188: mol_type = other DNA
atggtgcagcaggccgagagctcggaagccgagagcaacctgccccgggacgcgctggacaccgaggagggcgagttcatggcgtgcagcccggtggccctggacgagagcgacccggactggtgcaagacggcgtcgggccacatcaaacggcccatgaacgccttcatggtgtggtccaagatcgagcgcaggaagatcatggagcagtcgcccgacatgcacaacgccgagatctccaagaggctgggcaagcgctggaagatgctgaaggacagcgagaagatcccgttcatcagggaggcggagcgcctgcgcctcaagcacatggctgattatcccgactacaagtaccggccgcgcaaaaagcccaagacggacccagcggccaagcccagcgcgggccagagccccgacaagagcgcggcgggcgccaaggcagccaagggccccggcaagaagtgcgccaagctcaaggcgcctgcgggcaaggcgggcgcgggcaaggcggcgcagccgggggactgcgccgcgggcaaggcagccaagtgcgtcttcctggacgacgacgatgaagacgacgacgaagatgacgagctgcagctacggcccaagccggacgctgacgacgacgacgacgagcccgcgcactcgcacctgctgccgccgccgacgcagcagcaaccccctcagctgctgaggcgctacagcgtggccaaggtccccgccagccccacgctcagcagtgccgccgagtcccccgagggcgcgagcctgtacgacgaagtgcgcgcgggcggccggctctactacagcttcaagaacatcaccaagcagcagcctccgcccgcgcctcccgcgctgtcgcccgcgtcctcccgctgcgtgtccacctcctcatccagcggcagcagcagcggcagcggcgccgaggatgcagacgacctcatgttcgacctgagcttgaatttctcccagggcgcgcacagcgcctgcgagcagccactgggcgcgggagcggcggggaacctgtccctgtcgctggtggataaggacctggattccttcagcgagggcagcctgggttcccacttcgagttccccgactactgcacgccggagctgagcgagatgatcgcgggggactggctggaggcgaacttctccgacctggtgttcacgtattga

SEQ ID NO: 19 - protein, 349 aa, Homo sapiens
MGDMGDPPKKKRLISLCVGCGNQIHDQYILRVSPDLEWHAACLKCAECNQYLDESCTCFVRDGKTYCKRDYIRLYGIKCAKCSIGFSKNDFVMRARSKVYHIECFRCVACSRQLIPGDEFALREDGLFCRADHDVVERASLGAGDPLSPLHPARPLQMAAEPISARQPALRPHVHKQPEKTTRVRTVLNEKQLHTLRTCYAANPRPDALMKEQLVEMTGLSPRVIRVWFQNKRCKDKKRSIMMKQLQQQQPNDKTNIQGMTGTPMVAASPERHDGGLQANPVEVQSYQPPWKVLSDFALQSDIDQPAFQQLVNFSEGGPGSNSTGSEVASMSSQLPDTPNSMVASPIEA

SEQ ID NO: 20 - DNA, 1050 bp, Homo sapiens
  source 1..1050: mol_type = other DNA
atgggagacatgggagatccaccaaaaaaaaaacgtctgatttccctatgtgttggttgcggcaatcagattcacgatcagtatattctgagggtttctccggatttggaatggcatgcggcatgtttgaaatgtgcggagtgtaatcagtatttggacgagagctgtacatgctttgttagggatgggaaaacctactgtaaaagagattatatcaggttgtacgggatcaaatgcgccaagtgcagcatcggcttcagcaagaacgacttcgtgatgcgtgcccgctccaaggtgtatcacatcgagtgtttccgctgtgtggcctgcagccgccagctcatccctggggacgaatttgcgcttcgggaggacggtctcttctgccgagcagaccacgatgtggtggagagggccagtctaggcgctggcgacccgctcagtcccctgcatccagcgcggccactgcaaatggcagcggagcccatctccgccaggcagccagccctgcggccccacgtccacaagcagccggagaagaccacccgcgtgcggactgtgctgaacgagaagcagctgcacaccttgcggacctgctacgccgcaaacccgcggccagatgcgctcatgaaggagcaactggtagagatgacgggcctcagtccccgtgtgatccgggtctggtttcaaaacaagcggtgcaaggacaagaagcgaagcatcatgatgaagcaactccagcagcagcagcccaatgacaaaactaatatccaggggatgacaggaactcccatggtggctgccagtccagagagacacgacggtggcttacaggctaacccagtggaagtacaaagttaccagccaccttggaaagtactgagcgacttcgccttgcagagtgacatagatcagcctgcttttcagcaactggtcaatttttcagaaggaggaccgggctctaattccactggcagtgaagtagcatcaatgtcctctcaacttccagatacacctaacagcatggtagccagtcctattgaggcatga

SEQ ID NO: 21 - protein, 349 aa, Mus musculus; identical to SEQ ID NO: 19

SEQ ID NO: 22 - DNA, 1050 bp, Mus musculus
  source 1..1050: mol_type = other DNA
atgggagacatgggcgatccaccaaaaaaaaaacgtctgatttccctgtgtgttggttgcggcaatcaaattcacgaccagtatattctgagggtttctccggatttggagtggcatgcagcatgtttgaaatgtgcggagtgtaatcagtatttggacgaaagctgtacgtgctttgttagggatgggaaaacctactgtaaaagagattatatcaggttgtacgggatcaaatgcgccaagtgcagcataggcttcagcaagaacgacttcgtgatgcgtgcccgctctaaggtgtaccacatcgagtgtttccgctgtgtagcctgcagccgacagctcatcccgggagacgaattcgccctgcgggaggatgggcttttctgccgtgcagaccacgatgtggtggagagagccagcctgggagctggagaccctctcagtcccttgcatccagcgcggcctctgcaaatggcagccgaacccatctcggctaggcagccagctctgcggccgcacgtccacaagcagccggagaagaccacccgagtgcggactgtgctcaacgagaagcagctgcacaccttgcggacctgctatgccgccaaccctcggccagatgcgctcatgaaggagcaactagtggagatgacgggcctcagtcccagagtcatccgagtgtggtttcaaaacaagcggtgcaaggacaagaaacgcagcatcatgatgaagcagctccagcagcagcaacccaacgacaaaactaatatccaggggatgacaggaactcccatggtggctgctagtccggagagacatgatggtggtttacaggctaacccagtagaggtgcaaagttaccagccgccctggaaagtactgagtgacttcgccttgcaaagcgacatagatcagcctgcttttcagcaactggtcaatttttcagaaggaggaccaggctctaattctactggcagtgaagtagcatcgatgtcctcgcagctcccagatacacccaacagcatggtagccagtcctattgaggcatga

SEQ ID NO: 23 - DNA, 53 bp, synthetic construct
  misc_feature 1..53: note = Synthetic
  source 1..53: mol_type = other DNA
gcccacctgtagcatcatcaacatgggaccccagaacagaaacactagctttg

SEQ ID NO: 24 - DNA, 448 bp, Homo sapiens
  source 1..448: mol_type = other DNA
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 25 - DNA, 601 bp, Homo sapiens
  source 1..601: mol_type = other DNA
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattagcatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccgtttagtgaacg

SEQ ID NO: 26 - DNA, 601 bp, Homo sapiens
  source 1..601: mol_type = other DNA
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattagcatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtagggtgggaggtctatataagcagagctccgtttagtgaacg

SEQ ID NO: 27 - DNA, 601 bp, Homo sapiens
  source 1..601: mol_type = other DNA
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattagcatggtgatgcggttttggcagtacatcaatgggcgtggataggggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccgtttagtgaacg